(19) United States
(12) Patent Application Publication
(10) Pub. No.: US 2023/0332217 A1
(43) Pub. Date: Oct. 19, 2023

(54) METHOD FOR POSITIVE CONTROL REACTION USING PRE-POSITIVE CONTROL COMPOSITION

(71) Applicant: SEEGENE, INC., Seoul (KR)

(72) Inventors: Jihoon PARK, Hanam-si, Gyeonggi-do (KR); Soyoung KIM, Seoul (KR); Dong-Min KIM, Seoul (KR); Ohsung KO, Hanam-si, Gyeonggi-do (KR)

(73) Assignee: SEEGENE, INC., Seoul (KR)

(21) Appl. No.: 18/023,105

(22) PCT Filed: Aug. 27, 2021
(86) PCT No.: PCT/KR2021/011556

§ 371 Date: Feb. 24, 2023

(30) Foreign Application Priority Data
Aug. 28, 2020 (KR) 10-2020-0109386

(51) Int. Cl. C12Q 1/6853

(52) U.S. Cl. CPC C12Q 1/6853; C12Q 2600/166; C12Q 2600/16

(57) ABSTRACT

Disclosed herein is a method for a positive control reaction using a pre-positive control composition. Unlike conventional positive controls, the pre-positive control composition according to the present disclosure is provided in the pre-positive control composition form, but not in a complete positive control form, until an experiment preparation stage, and can produce a complete-positive control through a positive control reaction, whereby the contamination that could occur in an experiment preparation stage for positive control preparation and positive control reactions can be minimize and an examination can be made to see whether the contamination comes from the positive control or not.

### TECHNICAL FIELD

The present disclosure relates to a method for a positive control reaction using a pre-positive control composition.

### BACKGROUND ART

Molecular diagnostics is a rapidly growing field in the in vitro diagnostics market for early diagnosis of diseases. Among others, methods using nucleic acids are advantageously used in diagnosing causative genetic factors attributed to viral or bacterial infection, etc., on the basis of high specificity and sensitivity.

Most of the diagnostic methods using nucleic acids include amplification of target nucleic acids (e.g., viral or bacterial nucleic acids). Representative of nucleic acid amplification methods is polymerase chain reaction (PCR) which is accounted for by repeated cycles of denaturing double-stranded DNA, annealing oligonucleotide primers to a DNA template, and extending the primers with DNA polymerase (Mullis et al., U.S. Pat. Nos. 4,683,195, 4,683,202, and 4,800,159; and Saiki et al., (1985) Science 230, 1350-1354).

Suggested are various different nucleic acid amplification processes including LCR (Ligase Chain Reaction), SDA (Strand Displacement Amplification), NASBA (Nucleic Acid Sequence-Based Amplification), TMA (Transcription Mediated Amplification), and RCA (Rolling-Circle Amplification).

Gene synthesis is another applied field of nucleic acid amplification, as exemplified by assembly PCR in which a large oligonucleotide is assembled from multiple shorter oligonucleotide fragments (Willem P. C. Stemmer et al., Gene, 164: 49-53, 1995), and fusion PCR.

Very elaborate techniques are required for the PCR that is applied to various diagnostics. In conventional PCR, primers, polymerase, dNTPs, reaction buffers, and so on are aliquoted into multi-microtubes or multi-well plates according to steps to prepare mixtures and a target nucleic acid to be amplified is added to each tube where the amplification is achieved. Like this, various reagents are required to be individually divided into trace amounts for PCR-based diagnostic experiments. However, experimental errors take place in such procedures, resulting in incorrect results.

For example, a false-positive result may be brought about by cross-over contamination between samples (e.g., specimens) handled during PCR reaction, such as template contamination from adjacent wells, aerosol transmission, etc., or carry-over contamination in which a result of a previous experiment (e.g., an amplicon) contaminates a new PCR reaction via various routes. Furthermore, PCR instrument error or reagent degradation may bring about a false-negative result.

To ensure diagnostic accuracy and reliability from such problems, a positive control and a negative control that can confirm false negative and false positive results can be used.

However, there is another problem that a false positive result occurs due to contamination with a positive control used to ensure accuracy and reliability. Usually, a plasmid DNA into which a target nucleic acid sequence to be detected is inserted is used as a positive control. When a false positive result occurs, it is impossible to identify the contamination source, that is to see whether the false positive result comes from the positive control or other factors because the positive control has a sequence 100% identical to a target nucleic acid sequence as a rule.

In addition, contamination may be made by a positive control in the process of preparing a plasmid DNA positive control or mutation may take place on a positive control sequence in the subculture for plasmid DNA production.

There is therefore a need for development of a novel positive control that allows a minimum degree of contamination if present during the preparation of the positive control and the amplification of a target nucleic acid sequence, can identify a contamination source upon the generation of contamination by determining whether the contamination comes from an amplicon or the positive control, and is of improved stability, compared to conventional positive controls.

Throughout the description, a number of publications and patent documents are referenced and citations are provided in parentheses. The disclosures of the cited publications and patent documents are incorporated herein by reference in its entirety to more clearly describe this invention and the state of the art to which the present invention pertains.

### Disclosure of Invention

### Technical Problem

The present inventors have conducted intensive and thorough research into the development of a novel positive control that allows a minimum degree of contamination if present during the preparation of the positive control and the amplification of a target nucleic acid sequence, can identify a contamination source upon the generation of contamination by determining whether the contamination comes from an amplicon or the positive control, and is of improved stability, compared to conventional positive controls. As a result, the present inventors succeed in developing a pre-positive control composition comprising a pre-positive control oligonucleotide and a method for performing a positive control reaction for a target nucleic acid sequence by using same. Unlike conventional positive controls, the pre-positive control composition according to the present disclosure is provided in the pre-positive control composition form, but not in a complete positive control form, until an experiment preparation stage, and can produce a complete-positive control through a positive control reaction, whereby the contamination that could occur in an experiment preparation stage for positive control preparation and positive control reactions can be minimize and an examination can be made to see whether the contamination comes from the positive control or not.

An aspect of the present disclosure is to provide a method for conducting a positive control reaction for a target nucleic acid-detecting composition by using a pre-positive control composition.

Another aspect of the present disclosure is to provide a pre-positive control composition that allows the production of a complete-positive control designed to carry out a positive control reaction for a target nucleic acid-detecting composition.

A further aspect of the present disclosure is to provide a kit for conducting a positive control reaction for a target nucleic acid-detecting composition.

Other objects and advantages of the present invention will become apparent from the detailed description to follow taken in conjugation with the appended claims and drawings.

### Solution to Problem

An aspect of the present disclosure provides a method for conducting a positive control reaction for a target nucleic acid-detecting composition by using a pre-positive control composition, the method comprising the steps of:
(a) mixing the target nucleic acid-detecting composition and the pre-positive control composition, wherein, the target nucleic acid-detecting composition comprises target nucleic acid-detecting oligonucleotides comprising a forward primer oligonucleotide and a reverse primer oligonucleotide for target nucleic acid amplification;
the pre-positive control composition comprises a pre-positive control oligonucleotide, the pre-positive control oligonucleotide comprising a partial sequence of a complete-positive control oligonucleotide for the target nucleic acid sequence;
the pre-positive control oligonucleotide comprises a sequence overlapping with at least one oligonucleotide selected from among the target nucleic acid-detecting oligonucleotides and a different pre-positive control oligonucleotide, wherein the overlapping sequence of the pre-positive control oligonucleotide is identical or complementary to an overlapping sequence of the at least one oligonucleotide;
the pre-positive control composition comprises one or more pre-positive control oligonucleotides; and
the one or more pre-positive control oligonucleotides are selected so as to produce the complete-positive control by assembling (i) the pre-positive control oligonucleotides or (ii) the one or more pre-positive control oligonucleotides and the at least one of the target nucleic acid-detecting oligonucleotides;
(b) producing the complete-positive control oligonucleotide through an assembly process between different oligonucleotides, the assembly process comprising the steps of:
(b-1) hybridizing at least one complementary combination among combinations of two oligonucleotides comprising an overlapping sequence identical or complementary to each other in the mixture;
(b-2) extending either or both of the two oligonucleotides hybridized with each other; and
(b-3) producing the complete-positive control oligonucleotide;
wherein a product resulting from the extending step is the complete-positive control oligonucleotide or an intermediate-positive control oligonucleotide, and when the intermediate-positive control oligonucleotide is produced, at least one round of a step of denaturing double-stranded nucleic acids and steps (b-1) and (b-2) is further repeated to produce the complete-positive control oligonucleotide; and
(c) amplifying the complete-positive control oligonucleotide using the forward primer oligonucleotide and the reverse primer oligonucleotide.

The present inventors have conducted intensive and thorough research into the development of a novel positive control that allows a minimum degree of contamination if present during the preparation of the positive control and the amplification of a nucleic acid sequence, can identify a contamination source upon the generation of contamination by determining whether the contamination comes from an amplicon or the positive control, and is of improved stability, compared to conventional positive controls. As a result, the present inventors succeed in developing a pre-positive control composition comprising a pre-positive control oligonucleotide and a method for performing a positive control reaction for a target nucleic acid sequence by using same. Unlike conventional positive controls, the pre-positive control composition according to the present disclosure is provided in the pre-positive control composition form, but not in a complete positive control form, until an experiment preparation stage, and can produce a complete-positive control through a positive control reaction, whereby the contamination that could occur in an experiment preparation stage for positive control preparation and positive control reactions can be minimize and an examination can be made to see whether the contamination comes from the positive control or not.

Below, a detailed description will be given of the present disclosure with reference to the drawings:

Step (a): Mixing Target Nucleic Acid-Detecting Composition and Pre-Positive Control Composition

According to the present disclosure, first, a target nucleic acid-detecting composition and a pre-positive control composition are mixed with each other.

The term “nucleic acid”, “nucleic acid sequence”, or “nucleic acid molecule”, as used herein, refer to a deoxyribonucleotide or ribonucleotide polymer in a single- or double-stranded form. In this regard, the nucleotide includes a derivative of a naturally occurring nucleotide, a non-naturally occurring nucleotide, or a modified nucleotide which can function in the same manner as the naturally occurring nucleotide.

As used herein, the term “target nucleic acid”, “target nucleic acid sequence”, or “target sequence” refers to a nucleic acid sequence to be detected, which is annealed or hybridized with a primer or a probe in a hybridization, annealing, or amplification condition.

Examples of the target nucleic acid include nucleic acids of prokaryotes, eukaryotes (e.g., protozoa, parasites, fungi, yeasts, higher plants, lower animals, and higher animals including mammals and humans), viruses, and viroids. In addition, the target nucleic acid may be a nucleic acid having an artificially synthesized sequence.

In an embodiment of the present disclosure, the target nucleic acid-detecting composition may comprise one or more target nucleic acid-detecting oligonucleotides for use in amplifying or detecting a target nucleic acid.

In an embodiment of the present disclosure, the target nucleic acid-detecting oligonucleotides may comprise a forward primer oligonucleotide and a reverse primer oligonucleotide for target nucleic acid amplification.

As used herein, the term “primer” refers to an oligonucleotide that is capable of acting as a point of initiation of synthesis in a condition for inducing the synthesis of a primer extension product complementary to a target nucleic acid sequence (template), i.e., in a condition including the presence of nucleotides and an agent for polymerization, such as DNA polymerase, and suitable temperatures and pH. The primer should be long enough to prime the synthesis of an extension product in the presence of a polymerizing agent. A suitable length of the primer is determined depending on various factors including, for example, a temperature, an applied field, and a source thereof.

In an embodiment of the present disclosure, the target nucleic acid-detecting oligonucleotides may further comprise a target nucleic acid-detecting probe.

As used herein, the term “probe” refers to a single-stranded nucleic acid molecule including a portion or portions substantially complementary to a target nucleic acid sequence.

According to an embodiment of the present disclosure, the probe has a 3′-end that is “blocked” to prevent extension therefrom. The blocking may be achieved in accordance with conventional methods. For instance, the blocking may be performed by adding to the 3′-hydroxyl group of the last nucleotide a chemical moiety such as biotin, a label, a phosphate group, an alkyl group, a non-nucleotide linker, phosphorothioate, or an alkane-diol residue. Alternatively, the blocking may be carried out by removing the 3′-hydroxyl group of the last nucleotide or using a nucleotide lacking a 3′-hydroxyl group, like dideoxynucleotide.

The primer or the probe may be a single strand. The primer or the probe includes deoxyribonucleotides, ribonucleotides, or a combination thereof. The primer or the probe available in the present disclosure may include naturally occurring dNMPs (e.g., dAMP, dGMP, dCMP, and dTMP), modified nucleotides, or non-naturally occurring nucleotides.

The term “annealing” or “priming” as used herein refers to the apposition of an oligonucleotide or nucleic acid to a template nucleic acid, whereby the polymerase is allowed to polymerize nucleotides into a nucleic acid molecule which is complementary to the template nucleic acid or a portion thereof.

The term “hybridizing”, as used herein, refers to the formation of a double-stranded nucleic acid from two single-stranded polynucleotides through non-covalent bonds between complementary nucleotide sequences thereof in a predetermined hybridization condition.

In an embodiment of the present disclosure, the pre-positive control composition may comprise at least one pre-positive control oligonucleotide (pre-PC oligonucleotide).

In an embodiment of the present disclosure, the pre-positive control oligonucleotide may be a single- or double-stranded oligonucleotide.

In an embodiment of the present disclosure, the pre-positive control oligonucleotide may be a single-stranded oligonucleotide.

As used herein, the term “positive control” refers to an internal control substance or an external control substance which can act in a similar manner to that of the target nucleic acid.

Particularly, a positive control can be used in order to examine whether ingredients of the target nucleic acid-detecting composition work properly, for example, whether a primer and/or a probe normally acts for amplification and/or detection of a target nucleic acids. To this end, a conventional positive control includes a sequence complementary to a target nucleic acid-detecting oligonucleotide and can be amplified and/or detected using the target nucleic acid-detecting oligonucleotide (e.g., a primer and/or a probe) in a condition suitable for amplifying and detecting the target nucleic acid.

For example, a conventional positive control is one oligonucleotide in a single- or double-stranded form. The one oligonucleotide includes all sequences complementary to the target nucleic acid-detecting oligonucleotides included in the target nucleic acid-detecting composition.

According to an embodiment, the one oligonucleotide includes an entirety of the target nucleic acid sequence to be amplified with a pair of primers for amplifying the target nucleic acid.

According to another embodiment, the one oligonucleotide does not include an entirety of the target nucleic acid sequence, but all the sequences complementary to the target nucleic acid-detecting oligonucleotides in the target nucleic acid-detecting composition. For example, the one oligonucleotide may be designed to include only the complementary sequences among the entire target nucleic acid sequence. Alternatively, the one oligonucleotide may be designed to include the complementary sequence plus a part of sequences other than the complementary sequence among the entire target nucleic acid sequence.

As used herein, the expression “the one positive control in a single-stranded oligonucleotide form includes all of sequences complementary to the target nucleic acid-detecting oligonucleotides” may mean that the single-stranded oligonucleotide or a complementary sequence thereto includes all of the sequences complementary to the target nucleic acid-detecting oligonucleotides. For instance, a conventional positive control in a single-stranded oligonucleotide form and a complementary sequence thereof may include sequences complementary to full sequences of a forward and a reverse primer.

In contrast, the pre-positive control composition of the present disclosure, which is used as a positive control for the target nucleic acid-detecting composition, comprises at least one pre-positive control oligonucleotide, with a proviso that one pre-positive control oligonucleotide does not include all of sequences complementary to the target nucleic acid-detecting oligonucleotides.

Particularly, the pre-positive control composition of the present disclosure is prepared into a positive control including all of complementary sequences to target nucleic acid-detecting oligonucleotides (i.e., complete-positive control) by assembling a plurality of oligonucleotides, for example, two or more pre-positive control oligonucleotides, or one or more pre-positive control oligonucleotide and one or more target nucleic acid-detecting oligonucleotides through a positive control reaction.

With reference to FIG. 1, for example, the pre-positive control composition according to the present disclosure may comprise one pre-positive control oligonucleotide. In this regard, the one pre-positive control oligonucleotide may include sequences complementary to a portion of a forward primer and a portion of a reverse primer as shown in FIG. 1A (a) or may include sequences complementary to the entirety of one of a forward and a reverse primer and a portion of the other, as shown in FIG. 1A (b) or (c). When a probe is present, the one pre-positive control oligonucleotide may include a sequence complementary to the full sequence of the probe.

A probe may or may not exist for cases shown in FIG. 1, except for FIGS. 1B (d) and 1C (d). In FIG. 1, the directionality of the probe may be 5′-3′ or 3′-5′. The pre-positive control oligonucleotide in FIG. 1 may be in a single- or double-stranded form. For the single-stranded form, the pre-positive control oligonucleotide may be positioned in a 5′-3′ or 3′-5′ direction.

In another embodiment, when the pre-positive control composition comprises two pre-positive control oligonucleotides, the two pre-positive control oligonucleotides may individually include a sequence complementary to an entirety or portion of a forward and a reverse primer, as shown in FIG. 1B (a), (c), or (d).

Alternatively, as shown in FIG. 1B (b), at least one of the two pre-positive control oligonucleotides includes a sequence complementary to both a forward and a reverse primer, wherein the complementary sequence covers a portion of one of the primers (reverse primer in FIG. 1B (b)), but not the full sequence of both of the forward and the reverse primer. When a probe is present, the two pre-positive control oligonucleotides individually include a sequence complementary to an entirety or part of the sequence of the probe. In this regard, a pre-positive control oligonucleotide and/or the complete-positive control produced from an assembly of pre-positive control oligonucleotides include the full sequence of the probe.

As described above, the present disclosure is characterized by the pre-positive control composition in which a complementary sequence to the full sequence of a plurality of target nucleic acid-detecting oligonucleotides included in the target nucleic acid-detecting composition is not covered by one oligonucleotide (i.e., one pre-positive control oligonucleotide), in contrast to a conventional positive control in which one oligonucleotide covers the full sequence of target nucleic acid-detecting oligonucleotides.

Specifically, a complete-positive control oligonucleotide including all the sequences complementary to the entire sequences of the target nucleic acid-detecting oligonucleotides is produced only by a positive control reaction (i.e., assembling) between the target nucleic acid-detecting oligonucleotides and/or the pre-positive control oligonucleotides.

In an embodiment of the present disclosure, the pre-positive control oligonucleotide may include a partial sequence of the complete-positive control oligonucleotide for the target nucleic acid sequence.

As used herein, the term “complete-positive control oligonucleotide” refers to a positive control oligonucleotide including all of the sequences complementary to target nucleic acid-detecting oligonucleotides. An example of the complete-positive control oligonucleotide is one oligonucleotide in a single- or double-stranded form, which includes all the sequences complementary to the target nucleic acid-detecting oligonucleotides included in a target nucleic acid-detecting composition described above as an example of a conventional positive control.

The complete-positive control oligonucleotide may be designed to include an entirety of a target nucleic acid sequence to be amplified with a pair of primers for target nucleic acid amplification. Alternatively, the complete-positive control oligonucleotide may be minimally designed to include only the sequences complementary to the entire sequences of the target nucleic acid-detecting oligonucleotides included in the target nucleic acid-detecting composition. For example, when the target nucleic acid-detecting composition comprises a pair of primers for target nucleic acid amplification, the complete-positive control oligonucleotide produced according to the method of the present disclosure may be minimally designed to include the entire sequences of the pair of primers. In another embodiment, when the target nucleic acid-detecting composition comprises a pair of primers for target nucleic acid amplification and a probe for target nucleic acid detection, the complete-positive control oligonucleotide produced according to the method of the present disclosure may be minimally designed to include only the entire sequences of the pair of primers and the probe.

In an embodiment of the present disclosure, the oligonucleotide are each 5 to 10,000 bp, 5 to 5,000 bp, to 4,000 bp, 5 to 3,000 bp, 5 to 2,000 bp, 5 to 1,000 bp, 5 to 900 bp, 5 to 800 bp, 5 to 700 bp, 5 to 600 bp, to 500 bp, 5 to 400 bp, 5 to 300 bp, 5 to 200 bp, 5 to 150 bp, 5 to 100 bp, 5 to 90 bp, 5 to 80 bp, 5 to 70 bp, to 60 bp, 5 to 50 bp, 5 to 40 bp, 5 to 30 bp, 5 to 20 bp, 5 to 10 bp, 10 to 1,000 bp, 10 to 900 bp, 10 to 800 bp, 10 to 700 bp, 10 to 600 bp, 10 to 500 bp, 10 to 400 bp, 10 to 300 bp, 10 to 200 bp, 10 to 150 bp, 10 to 100 bp, 10 to 90 bp, 10 to 80 bp, 10 to 70 bp, 10 to 60 bp, to 50 bp, 10 to 40 bp, 10 to 30 bp, 10 to 20 bp, 15 to 1,000 bp, 15 to 900 bp, 15 to 800 bp, 15 to 700 bp, to 600 bp, 15 to 500 bp, 15 to 400 bp, 15 to 300 bp, to 200 bp, 15 to 150 bp, 15 to 100 bp, 15 to 90 bp, to 80 bp, 15 to 70 bp, 15 to 60 bp, 15 to 50 bp, 15 to 40 bp, or 15 to 30 bp in length, but is not limited thereto.

In an embodiment of the present disclosure, the pre-positive control oligonucleotide may include a sequence overlapping with at least one oligonucleotide selected from among the target nucleic acid-detecting oligonucleotides and a different pre-positive control oligonucleotide, wherein the overlapping sequence of the pre-positive control oligonucleotide is identical or complementary to an overlapping sequence of the at least one oligonucleotide.

As used herein, the term “overlapping sequence” refers to an identical or complementary sequence between two oligonucleotides.

As for the overlapping sequence, the term “identical sequence” or “complementary sequence” mean a substantially identical sequence or a substantially complementary sequence as well as a completely identical sequence or a completely complementary sequence.

In the context of the overlapping sequence, the term “substantially identical sequence” means an identical sequence but with difference in some nucleotides, for example, 1, 2, 3, 4, 5, 6, 7, or 8 nucleotides.

In the context of the overlapping sequence, the term “substantially complementary sequence” means a complementary sequence, but with non-complementarity in some nucleotides, for example, 1, 2, 3, 4, 5, 6, 7, or 8 nucleotides.

In an embodiment of the present disclosure, the one or more pre-positive control oligonucleotides may include a sequence complementary to at least one target nucleic acid-detecting oligonucleotide. By way of example, when the pre-positive control composition comprises only one pre-positive control oligonucleotide, the one pre-positive control oligonucleotide may include a sequence overlapping with at least one target nucleic acid-detecting oligonucleotide. Alternatively, when the pre-positive control composition comprises two or more pre-positive control oligonucleotides, at least one of the two or more pre-positive control oligonucleotides may include a sequence overlapping with at least one target nucleic acid-detecting oligonucleotide.

In an embodiment of the present disclosure, the at least one pre-positive control oligonucleotide may include a sequence overlapping with at least one of the forward and the reverse primer oligonucleotides for target nucleic acid amplification. For instance, when the pre-positive control composition comprises only one pre-positive control oligonucleotide, the one pre-positive control oligonucleotide may include a sequence overlapping with at least one of the forward and the reverse primer oligonucleotide for target nucleic acid amplification. In another example, when the pre-positive control composition comprises two or more pre-positive control oligonucleotides, at least one of the two or more pre-positive control oligonucleotides may include a sequence overlapping with at least one of the forward and the reverse primer oligonucleotide for target nucleic acid amplification.

In an embodiment of the present disclosure, when the pre-positive control composition comprises only one pre-positive control oligonucleotide, the one pre-positive control oligonucleotide may include sequences overlapping with the forward and the reverse primer oligonucleotide for target nucleic acid amplification at its 3′-end and 5′-end, respectively (see FIG. 1A).

In an embodiment of the present disclosure, when the pre-positive control composition comprises two or more pre-positive control oligonucleotides, two of the two or more pre-positive control oligonucleotides include sequences overlapping with the forward and the reverse primer oligonucleotide for target nucleic acid amplification, respectively (see FIG. 1B).

In an embodiment of the present disclosure, a pre-positive control oligonucleotide may include a sequence overlapping with a different pre-positive control oligonucleotide or two different pre-positive control oligonucleotides.

An overlapping sequence between pre-positive control oligonucleotides or between a pre-positive control oligonucleotide and a target nucleic acid-detecting oligonucleotide may be utilized in an assembly process for producing a complete-positive control oligonucleotide.

In an embodiment of the present disclosure, an overlapping sequence between pre-positive control oligonucleotides or between a pre-positive control oligonucleotide and a target nucleic acid-detecting oligonucleotide may be selected so as to produce a complete-positive control oligonucleotide in an assembly process.

In an embodiment of the present disclosure, an overlapping sequence that one oligonucleotide has with respect to a different oligonucleotide may be a partial or full sequence of the one oligonucleotide.

In an embodiment, an overlapping sequence between two oligonucleotides is accounted for by individual partial sequences of the oligonucleotides.

In an embodiment, an overlapping sequence between a target nucleic acid-detecting oligonucleotide and a pre-positive control oligonucleotide may be accounted for by the full sequence of any one oligonucleotide.

In an embodiment, an overlapping sequence between pre-positive control oligonucleotides is not completely covered by any one of the pre-positive control oligonucleotides.

In an embodiment of the present disclosure, as shown for the forward primer oligonucleotide and the pre-positive control oligonucleotides in FIG. 1B, an overlapping sequence may be a partial sequence positioned at the 5′-end or 3′-end of the two oligonucleotides comprising the overlapping sequence.

According to another embodiment of the present disclosure, the overlapping sequence between two oligonucleotides may cover a partial sequence of one oligonucleotide and the full sequence of the other oligonucleotide. With reference to FIG. 1B (b), an overlapping sequence, if existing between a forward primer oligonucleotide and a pre-positive control oligonucleotide, is accounted for by a end portion of the pre-positive control oligonucleotide, and by the full sequence of the forward primer oligonucleotide.

In an embodiment of the present disclosure, a pre-positive control oligonucleotide may include overlapping with two different oligonucleotides at its 3′-end and 5′-end, respectively.

In an embodiment of the present disclosure, the overlapping sequence may be 3 to 100 bp, 3 to 90 bp, 3 to 80 bp, 3 to 70 bp, 3 to 60 bp, 3 to 50 bp, 3 to 40 bp, 3 to 30 bp, 3 to 20 bp, 3 to 10 bp, 3 to 5 bp, 5 to 100 bp, 5 to 90 bp, 5 to 80 bp, 5 to 70 bp, 5 to 60 bp, to 50 bp, 5 to 40 bp, 5 to 30 bp, 5 to 20 bp, 5 to 10 bp, 7 to 100 bp, 7 to 90 bp, 7 to 80 bp, 7 to 70 bp, 7 to 60 bp, 7 to 50 bp, 7 to 40 bp, 7 to 30 bp, 7 to 20 bp, 7 to 10 bp, 10 to 100 bp, 10 to 90 bp, 10 to 80 bp, to 70 bp, 10 to 60 bp, 10 to 50 bp, 10 to 40 bp, 10 to 30 bp, 10 to 20 bp, 10 to 15 bp, 15 to 100 bp, 15 to 90 bp, 15 to 80 bp, 15 to 70 bp, 15 to 60 bp, 15 to 50 bp, 15 to 40 bp, 15 to 30 bp, or 15 to 20 bp in length, but is not limited thereto.

As used herein, the term “assembly” refers to a reaction by which two or more oligonucleotides are joined to each other via an overlapping sequence therebetween to produce a longer oligonucleotide. For example, two or more oligonucleotides (e.g., pre-positive control oligonucleotide and/or target nucleic acid-detecting oligonucleotides) may be assembled into a longer oligonucleotide (complete-positive control oligonucleotide), using assembly PCR (Willem P. C. Stemmer et al., Gene, 164: 49-53, 1995).

In an embodiment of the present disclosure, the overlapping sequence between two oligonucleotides may be an identical or complementary sequence therebetween.

In an embodiment, when the overlapping sequence is an identical sequence between two oligonucleotides, a complementary strand may be produced with any one of the two oligonucleotides serving as a template, after which the synthesized complementary strand may be hybridized with the overlapping sequence on the other oligonucleotide and extended to complete the assembly (see FIG. 2A).

In an embodiment, when the overlapping sequence is a complementary sequence between two oligonucleotides, the two oligonucleotides (for example, pre-PC TO-1 and pre-PC TO-2 in FIG. 2B) may be hybridized through the overlapping sequence and extended to complete the assembly.

In an embodiment of the present disclosure, when two oligonucleotides have overlapping sequences complementary to each other wherein the complementary overlapping sequences are located at 5′-ends of both the oligonucleotides, the two oligonucleotides can be hybridized to each other through the overlapping sequences, but is impossible to extend. In this regard, with reference to FIG. 2C, the two oligonucleotides (e.g., pre-PC TO-1 and pre-PC TO-2 in FIG. 2C) are used as templates to produce strands complementary thereto with the aid of different oligonucleotides (e.g., forward primer and reverse primer in FIG. 2C) overlapping with 3′-end portions thereof, respectively, after which the produced complementary strands are hybridized to each other via an overlapping sequence therebetween and extended to complete the assembly.

In an embodiment of the present disclosure, when an overlapping sequence between two oligonucleotides is accounted for by a partial sequence of each of the two oligonucleotides, the two oligonucleotides can be joined to each other via the overlapping sequence and assembled to a longer oligonucleotide.

In another embodiment of the present disclosure, when an overlapping sequence between two oligonucleotides covers a partial sequence of one of the two oligonucleotides and a full sequence of the other, the two oligonucleotides may be hybridized to each other via the overlapping sequence and extended to afford an extension product (e.g., extended oligonucleotide or extended duplex). In this regard, the extension production may be an assembled oligonucleotide that is not longer than the full length. In this case, the extension product may be assembled with a different oligonucleotide to a longer oligonucleotide. For example, as shown in FIG. 1B (b), when there is a complementary sequence between a forward primer and a pre-positive control oligonucleotide, the two oligonucleotides can be hybridized to each other and extended. However, the extension production resulting from the assembly of the two oligonucleotides is not longer than, but as long as the pre-positive control oligonucleotide.

Hence, the term “assembly” is intended to encompass not only a reaction resulting in a longer oligonucleotide, but also an intermediate reaction for producing a longer oligonucleotide (e.g., reaction to afford an extension production, which is one of intermediate positive control oligonucleotides).

In an embodiment of the present disclosure, sequences of the different pre-positive control oligonucleotides may be selected such that assembly of the different pre-positive control oligonucleotides results in a complete-positive control oligonucleotide for target nucleic acid sequence. In this context, the assembly into complete-positive control oligonucleotide can be achieved with only the pre-positive control composition even if none of the target nucleic acid-detecting oligonucleotides exist. For example, as shown in FIG. 2B, when there is a complementary overlapping sequence between two pre-positive control oligonucleotides which cover full sequences of a forward and a reverse primer, respectively, a complete-positive control composition can be produced even by assembly of the two pre-positive control oligonucleotides only.

In an embodiment of the present disclosure, the 3′-end of the pre-positive control oligonucleotide may be in an extendable state.

In another embodiment of the present disclosure, the pre-positive control oligonucleotide may be blocked to prevent extension therefrom.

The blocking may be achieved according to a typical method. By way of example, the blocking may be conducted by adding to 3′-hydroxy on the last nucleotide a chemical moiety such as biotin, a label, a phosphate group, an alkyl group, a non-nucleotide linker, phosphorothioate, or an alkane-diol residue. Alternatively, the blocking may be conducted by removing 3′-hydroxy on the last nucleotide or using a nucleotide free of 3′-hydroxy, such as dideoxynucleotide.

In an embodiment of the present disclosure, the sequence of the pre-positive control oligonucleotide may be selected such that a complete-positive control oligonucleotide for a target nucleic acid sequence can be produced when at least one of one or more target nucleic acid-detecting oligonucleotides is assembled with the pre-positive control oligonucleotide.

In an embodiment of the present disclosure, the pre-positive control oligonucleotide may be a pre-positive control templating oligonucleotide (pre-PC TO) or a pre-positive control linking oligonucleotide (pre-PC LO).

According to an embodiment of the present disclosure, the pre-positive control templating oligonucleotide includes a sequence overlapping with at least one of the forward primer oligonucleotide and the reverse primer oligonucleotide, but may not include a sequence overlapping totally with the full sequences of the forward primer oligonucleotide and the reverse primer oligonucleotide.

According to an embodiment of the present disclosure, the pre-positive control linking oligonucleotide may include sequences overlapping with two different pre-positive control oligonucleotides at its 3′-end and 5′-end and may not include a sequence overlapping with the forward primer oligonucleotide or the reverse primer oligonucleotide. The pre-positive control linking oligonucleotide may join two different pre-positive control oligonucleotides to each other to achieve an assembly into a longer oligonucleotide.

In addition, when a probe is present in the mixture, the pre-positive control linking oligonucleotide may include a sequence identical or complementary to an entirety or portion of the probe. Particularly in the case of FIG. 1C (d), the pre-positive control linking oligonucleotide may include a sequence overlapping with the probe and act to join the probe and different pre-positive control oligonucleotides to make assembly into a longer oligonucleotide. In this regard, the 3′-end of the probe may be in an extendable state or may be blocked to prevent extension therefrom.

In an embodiment of the present disclosure, the pre-positive control composition may comprise one pre-positive control oligonucleotide wherein the pre-positive control oligonucleotide may include one pre-positive control templating oligonucleotide.

For example, as shown in FIG. 1A, the one pre-positive control templating oligonucleotide may include sequences overlapping with the forward primer oligonucleotide and reverse primer oligonucleotide at its 3′-end and 5′-end, respectively and may not include sequences overlapping totally with the full sequences of the forward primer oligonucleotide and the reverse primer oligonucleotide.

The pre-positive control templating oligonucleotide, if in a single-stranded form, may be hybridized with any one primer including a complementary overlapping sequence therewith, which may be either of a forward primer and a reverse primer depending on the directionality of the pre-positive control templating oligonucleotide, and then extended to produce an intermediate-positive control oligonucleotide. The produced intermediate-positive control oligonucleotide is separated into two single strands through double-stranded nucleic acid denaturation. The separated strand is hybridized with the other primer through a complementary overlapping sequence therebetween and extended to finally produce a complete-positive control oligonucleotide.

In an embodiment, when the pre-positive control composition comprises two or more pre-positive control oligonucleotides, the two or more pre-positive control oligonucleotides may include two pre-positive control templating oligonucleotides. The two pre-positive control templating oligonucleotides include a first and a second pre-positive control templating oligonucleotide that include overlapping sequences complementary to the forward and the reverse primer oligonucleotide, respectively.

In an embodiment, the pre-positive control composition comprises two pre-positive control oligonucleotides that may include two pre-positive control templating oligonucleotides. For example, as shown in FIG. 1B (a)-(c), the two pre-positive control templating oligonucleotides each include a sequence overlapping with the forward or the reverse primer oligonucleotide at one end thereof and an overlapping sequence therebetween at the other end thereof. For FIG. 1B (a), the forward primer and the reverse primer should be assembled, together with the pre-positive control oligonucleotides, to produce a complete-positive control. In case of FIG. 1B (b), the pre-positive control oligonucleotides should be assembled, together the reverse primer, to produce a complete-positive control. On the other hand, for FIG. 1B (c), a complete-positive control can be produced with only the pre-positive control oligonucleotides.

When the target nucleic acid-detecting composition comprises a probe, the two pre-positive control templating oligonucleotides may include a complementary or identical sequence to a partial or full sequence of the probe.

For instance, as shown in FIG. 1B (a) or (b), at least one of the two pre-positive control oligonucleotides may include the full sequence of the probe. Alternatively, as shown in FIG. 1B (c), the two pre-positive control oligonucleotides may each include a partial sequence of the probe such that the complete-positive control produced through the assembly of the pre-positive control composition can include the full sequence of the probe.

In another embodiment, as shown in FIG. 1B (d), the probe may act like a pre-positive control oligonucleotide and as such, can be involved in producing a complete-positive control oligonucleotide. In this regard, the pre-positive control oligonucleotide should be assembled together with the probe acting like a pre-positive control oligonucleotide, particularly a pre-positive control linking oligonucleotide, as well as the forward and the reverse primer, to produce a complete-positive control.

In an embodiment, the pre-positive control composition may three pre-positive control oligonucleotides, wherein the three pre-positive control oligonucleotides may include two pre-positive control templating oligonucleotides and one pre-positive linking oligonucleotide.

For example, as shown in FIG. 1C (a), (b), or (c), the two pre-positive control templating oligonucleotides may include sequences at one end thereof which overlap with the forward primer oligonucleotide and the reverse primer oligonucleotide, respectively, and may include sequences at the other end thereof, which overlap with 3′-end and 5′-end sequences of the pre-positive control linking oligonucleotide, respectively.

When the target nucleic acid-detecting composition comprises a probe, the three pre-positive control oligonucleotide may each include a sequence complementary or identical to a partial or full sequence of the probe sequence. For example, as shown in FIG. 1C (a) or (b), at least one of the three pre-positive control oligonucleotides may include the full sequence of the probe. Particularly, when the pre-positive control linking oligonucleotide among the three pre-positive control oligonucleotides includes the full sequence of the probe, the pre-positive control linking oligonucleotide may be designed to include an identical sequence to the probe (i.e., the same directionality as the probe) to prevent the probe and the pre-positive control linking oligonucleotide from competing for hybridization with the other pre-positive control oligonucleotide in the assembly process for a complete-positive control oligonucleotide.

In another example, as shown in FIG. 1B (c), the three pre-positive control oligonucleotides may each include a partial sequence of the probe so that the complete-positive control includes the full sequence of the probe.

In a further example, as shown in FIG. 1C (d), the probe may act like a pre-positive control oligonucleotide and as such, may be involved in producing a complete-positive control oligonucleotide. the pre-positive control oligonucleotide should be assembled together with the probe acting like a pre-positive control oligonucleotide, particularly a pre-positive control linking oligonucleotide, as well as the forward and the reverse primer, to produce a complete-positive control.

In an embodiment of the present disclosure, the 3′-end of the probe may be in an extendable state therefrom.

In an embodiment of the present disclosure, the probe may be selected so as to act like a pre-positive control oligonucleotide to produce a complete-positive control oligonucleotide (see FIGS. 1B (d) and 1C (d)).

In an embodiment, the pre-positive control composition may comprise four pre-positive control oligonucleotides which include two pre-positive control templating oligonucleotides and two pre-positive linking oligonucleotides. For example, one of the two pre-positive control templating oligonucleotides may include a sequence overlapping with a forward primer oligonucleotide at one end thereof and a sequence overlapping with one of the two pre-positive linking oligonucleotides at the other end thereof while the other pre-positive control templating oligonucleotide may include a sequence overlapping with a reverse primer oligonucleotide at one end thereof and a sequence overlapping with the other pre-positive linking oligonucleotide at the other end thereof. The two pre-positive control linking oligonucleotides may include the sequences overlapping respectively with the two pre-positive control templating oligonucleotides at their individual one end, as described above, and may include an overlapping sequence therebetween at their individual the other end (see FIG. 1D (a)).

A complete-positive control produced from the pre-positive control composition according to the present disclosure includes all of the complementary sequences to the full sequences of the target nucleic acid-detecting oligonucleotides (e.g., a pair of primers and/or probe). On the other hand, any one of the pre-positive control oligonucleotides included in the pre-positive control composition does not include all the sequences overlapping totally with the full sequence of the forward primer oligonucleotide and the full sequence of the reverse primer oligonucleotide.

As described above, the pre-positive control composition according to the present disclosure includes one or more pre-positive control oligonucleotides. The number of the pre-positive control oligonucleotides may be various. For example, one or more, two or more, three or more, four or more, five or more, six or more, seven or more, eight or more, 10 or more, 20 or more, or 30 or more pre-positive control oligonucleotides may be included, but with no limitations thereto.

In an embodiment of the present disclosure, the pre-positive control composition may comprise 50 or less, 40 or less, 30 or less, 20 or less, 10 or less, 9 or less, 8 or less, 7 or less, 6 or less, 5 or less, or 4 or less pre-positive control oligonucleotides.

In an embodiment of the present disclosure, the pre-positive control composition may comprise 1 to 5 pre-positive control oligonucleotides.

In an embodiment of the present disclosure, when the pre-positive control composition comprises 4 or more pre-positive control oligonucleotides, for example, n pre-positive control oligonucleotides (n≥4), the pre-positive control composition comprises 2 pre-positive control templating oligonucleotides and (n−2) pre-positive control linking oligonucleotides.

In an embodiment, the concentration of the pre-positive control oligonucleotide (e.g., pre-positive control templating oligonucleotide) is sufficient to produce a complete-positive control oligonucleotide, but may be not sufficient to amplify and/or detect the complete-positive control oligonucleotide.

In an embodiment, a larger number of the pre-positive control oligonucleotides in the pre-positive control composition may require a greater minimum concentration of the pre-positive control oligonucleotides which is necessary for producing a pre-positive control composition. For example, a minimum concentration necessary to produce a complete-positive control oligonucleotide may be larger for the employment of three pre-positive control oligonucleotides than two pre-positive control oligonucleotides.

In an embodiment, the pre-positive control composition may comprise the pre-positive control oligonucleotide at a concentration of 100 zmol/μl to 100 pmol/μl, 100 zmol/μl to 50 pmol/μl, 100 zmol/μl to 20 pmol/μl, 1 amol/μl to 100 pmol/μl, 1 amol/μl to 50 pmol/μl, or 1 amol/μl to 20 pmol/μl.

Alternatively, the pre-positive control composition may comprise 1×10⁴ copies to 1×10¹⁵ copies, 1×10⁵ copies to 1×10¹⁵ copies, 1×10⁶ copies to 1×10¹⁵ copies, or 1×10⁷ copies to 1×10¹⁵ copies of the pre-positive control oligonucleotide per positive control reaction.

In an embodiment, when the pre-positive control composition comprises two or more pre-positive control oligonucleotides, the two or more pre-positive control oligonucleotides may be identical to or different from each other in terms of concentration. For example, when the pre-positive control composition comprises three pre-positive control oligonucleotides, the three pre-positive control oligonucleotides are all identical in concentration, two of the three pre-positive control oligonucleotides may have the same concentration and be different from the other in concentration, or the three pre-positive control oligonucleotides are different in concentration from one another.

In an embodiment, the concentration of the pre-positive control oligonucleotide may be lower than that at which the target nucleic acid-detecting oligonucleotides are used to amplify and/or detect a target nucleic acid.

In an embodiment, the pre-positive control oligonucleotide (e.g., pre-positive control templating oligonucleotide) may be included at a concentration 3-fold or less, 5-fold or less, 10-fold or less, 100-fold or less, 1,000-fold or less, 10,000-fold or less, 20,000-fold or less, 30,000-fold or less, 40,000-fold or less, or 50,000-fold or less than that of the target nucleic acid-detecting oligonucleotides (e.g., primer).

In an embodiment of the present disclosure, the complete-positive control oligonucleotide produced according to the present disclosure may be a single- or double-stranded oligonucleotide.

In an embodiment of the present disclosure, the complete-positive control oligonucleotide produced according to the present disclosure may be a double-stranded oligonucleotide.

In an embodiment, the sequence of the pre-positive control oligonucleotide may be selected such that a complete-positive control oligonucleotide can be produced for a target nucleic acid sequence when at least one of the one or more target nucleic acid-detecting oligonucleotides and the pre-positive control oligonucleotide are assembled together.

For instance, selection may be made such that the target nucleic acid-detecting oligonucleotide acts like a pre-positive control oligonucleotide to produce a complete-positive control oligonucleotide.

This is described in detail with reference to FIG. 2E. In step (a) of FIG. 2E, a target nucleic acid-detecting composition and a pre-positive control composition are mixed with each other and in step (b) of FIG. 2E, a forward primer oligonucleotide is assembled with a pre-positive control oligonucleotide (pre-PC TO-1) to produce an intermediate-positive control oligonucleotide. Subsequently, in step (c) of FIG. 2E, the intermediate-positive control oligonucleotide produced by assembly between the forward primer oligonucleotide and the pre-positive control oligonucleotide is assembled with a different intermediate-positive control oligonucleotide produced by assembly between two different pre-positive control oligonucleotides (pre-PC LO and pre-PC TO-2), whereby a complete-positive control oligonucleotide is obtained.

According to the present disclosure, as for the complete-positive control produced by a positive control reaction using the pre-positive control composition, its desired sequence may be designed by making an optimal selection from various combinations of sequences and number of pre-positive control nucleotides. Among others, a complete-positive control is designed to have a sequence different from that of an amplicon of a target nucleic acid, whereby if contamination occurs due to the complete-positive control, the contamination source can be discriminated.

In an embodiment of the present disclosure, the complete-positive control oligonucleotide produced from the pre-positive control composition may include an additional sequence plus the complementary sequences to the target nucleic acid-detecting oligonucleotides.

When a complete-positive control oligonucleotide produced from the pre-positive control composition sequentially includes complementary sequences to the target nucleic acid-detecting oligonucleotides, e.g., the forward primer, the probe, and the reverse primer, hybridization of the complete-positive control oligonucleotide with the forward primer, the probe, and the reverse primer may be disturbed. The sequential presence of the complementary sequences may be a barrier to suitable enzymatic association and reaction. This problem can be solved through a design made to include an additional sequence between complementary sequences to a plurality of target nucleic acid-detecting oligonucleotides in the complete-positive control oligonucleotide (that is, sequences hybridizable with target nucleic acid-detecting oligonucleotides).

In addition, the additional sequence may be a sequence that allows discrimination between a complete-positive control oligonucleotide produced from the pre-positive control composition and a target nucleic acid sequence.

The pre-positive control oligonucleotide may include the additional sequence.

The additional sequence may be a part of a target nucleic acid sequence except for the target nucleic acid-detecting oligonucleotides, or an artificially selected sequence.

The additional sequence is selected so as not to hybridize with the target nucleic acid-detecting oligonucleotides. In an embodiment, the additional sequence is a non-complementary sequence to the target nucleic acid-detecting oligonucleotides.

By way of example, the pre-positive control oligonucleotide may further include a non-complementary sequence to the target nucleic acid-detecting oligonucleotide plus complementary sequences to the target nucleic acid-detecting oligonucleotides so that a complete-positive control including the additional sequence in addition to complementary sequences to the target nucleic acid-detecting oligonucleotides can be produced through assembly between the pre-positive control oligonucleotide and/or the target nucleic acid-detecting oligonucleotides.

In an embodiment of the present disclosure, when the target nucleic acid-detecting oligonucleotides include a pair of primers, the pre-positive control oligonucleotide may include complementary sequences to the pair of forward and reverse primers and a non-complementary sequence to the target nucleic acid-detecting oligonucleotides.

In another embodiment of the present disclosure, when the target nucleic acid-detecting oligonucleotides include a pair of primers and a probe, the pre-positive control oligonucleotide may further include complementary sequences to the forward primer, the reverse primer and/or the probe, and a non-complementary sequence to the target nucleic acid-detecting oligonucleotides.

In another embodiment of the present disclosure, the pre-positive control oligonucleotide, especially, the pre-positive control linking oligonucleotide may be entirely selected as a non-complementary sequence to the target nucleic acid-detecting oligonucleotides.

As used herein, the term “non-complementary” refers to pertaining to sufficient lack of complementarity to allow selective hybridization with none of the pre-positive control oligonucleotide, the complete-positive control oligonucleotide, and complementary sequences thereto in a given annealing condition or stringent condition and is intended to encompass the terms “substantially non-complementary” and “perfectly noncomplementary”, with preference for perfectly noncomplementary.

The additional sequence may be 1 to 1,000 bp, 1 to 900 bp, 1 to 800 bp, 1 to 700 bp, 1 to 600 bp, 1 to 500 bp, 1 to 400 bp, 1 to 300 bp, 1 to 200 bp, 1 to 150 bp, 1 to 100 bp, 1 to 90 bp, 1 to 80 bp, 1 to 70 bp, 1 to 60 bp, 1 to 50 bp, 1 to 40 bp, 1 to 30 bp, 1 to 20 bp, 1 to 10 bp, 1 to 5 bp, 2 to 1,000 bp, 2 to 900 bp, 2 to 800 bp, 2 to 700 bp, 2 to 600 bp, 2 to 500 bp, 2 to 400 bp, 2 to 300 bp, 2 to 200 bp, 2 to 150 bp, 2 to 100 bp, 2 to 90 bp, 2 to 80 bp, 2 to 70 bp, 2 to 60 bp, 2 to 50 bp, 2 to 40 bp, 2 to 30 bp, 2 to 20 bp, 2 to 10 bp, 2 to 5 bp, 3 to 1,000 bp, 3 to 900 bp, 3 to 800 bp, 3 to 700 bp, 3 to 600 bp, 3 to 500 bp, 3 to 400 bp, 3 to 300 bp, 3 to 200 bp, 3 to 150 bp, 3 to 100 bp, 3 to 90 bp, 3 to 80 bp, 3 to 70 bp, 3 to 60 bp, 3 to 50 bp, 3 to 40 bp, 3 to 30 bp, 3 to 20 bp, 3 to 10 bp, 5 to 1,000 bp, 5 to 900 bp, 5 to 800 bp, 5 to 700 bp, 5 to 600 bp, 5 to 500 bp, to 400 bp, 5 to 300 bp, 5 to 200 bp, 5 to 150 bp, 5 to 100 bp, 5 to 90 bp, 5 to 80 bp, 5 to 70 bp, 5 to 60 bp, to 50 bp, 5 to 40 bp, 5 to 30 bp, 5 to 20 bp, 5 to 10 bp, 10 to 1,000 bp, 10 to 900 bp, 10 to 800 bp, 10 to 700 bp, 10 to 600 bp, 10 to 500 bp, 10 to 400 bp, 10 to 300 bp, 10 to 200 bp, 10 to 150 bp, 10 to 100 bp, 10 to 90 bp, 10 to 80 bp, 10 to 70 bp, 10 to 60 bp, 10 to 50 bp, 10 to 40 bp, 10 to 30 bp, 10 to 20 bp, 30 to 1,000 bp, 30 to 900 bp, 30 to 800 bp, 30 to 700 bp, 30 to 600 bp, 30 to 500 bp, 30 to 400 bp, 30 to 300 bp, 30 to 200 bp, 30 to 150 bp, 30 to 100 bp, or 30 to 50 bp long, but with no limitations thereto.

In the present disclosure, the target nucleic acid-detecting composition may optionally comprise reagents necessary for target amplification (e.g., PCR), including a nucleic acid polymerase, a buffer, a polymerase cofactor, and deoxyribonucleotide-5-triphosphate. Optionally, the target nucleic acid-detecting composition may also comprise various poly nucleotide molecules, reverse transcription enzyme, various buffers and reagents, and an inhibitory antibody against nucleic acid polymerase. Optimal amounts of reagents used for a specific reaction could be easily determined by a skilled person who understands the advantage of the present disclosure. Components of the nucleic acid-detecting composition may be included in individual containers or in combination in a single container.

Step (b): Production of Complete-Positive Control Oligonucleotide Through Assembly

The present disclosure comprises step (b) of producing the complete-positive control oligonucleotide through an assembly process between different oligonucleotides, the assembly process comprising the steps of:
(b-1) hybridizing at least one complementary combination among combinations of two oligonucleotides comprising an overlapping sequence identical or complementary to each other in the mixture;
(b-2) extending either or both of the two oligonucleotides hybridized with each other; and
(b-3) producing the complete-positive control oligonucleotide;
wherein a product resulting from the extending step is the complete-positive control oligonucleotide or an intermediate-positive control oligonucleotide, and when the intermediate-positive control oligonucleotide is produced, at least one round of a step of denaturing double-stranded nucleic acids and steps (b-1) and (b-2) is further repeated to produce the complete-positive control oligonucleotide.

Below, a detailed description will be given of step (b) with reference to FIG. 2:

(b-1) Hybridization of Two Oligonucleotides Including Complementary Overlapping Sequences to Each Other

The mixture of the pre-positive control composition and the target nucleic acid-detecting composition comprises two oligonucleotide combinations including identical overlapping sequences to each other (hereinafter referred to as “identical combination”) and/or two oligonucleotide combinations including complementary overlapping sequences to each other (hereinafter referred to as “complementary combination”).

In step (b-1), at least one complementary combination among combinations of two oligonucleotides comprising an overlapping sequence identical or complementary to each other is hybridized in the mixture;

In the case of FIG. 2E, among two primer oligonucleotides which are all the target nucleic acid-detecting oligonucleotides and three pre-positive control oligonucleotides, for example, two complementary combinations, that is, (i) a complementary combination of the forward primer oligonucleotide and the pre-positive control templating oligonucleotide (pre-PC TO-1) and (ii) a complementary combination of two pre-positive control oligonucleotides (pre-positive control linking oligonucleotide (pre-PC LO) and pre-positive control templating oligonucleotide (pre-PC TO-2)) exist, and at least one of the two complementary combinations is subjected to hybridization.

In an embodiment of the present disclosure, the combinations include overlapping sequences that are not identical or complementary to each other. In other words, when a plurality of combinations, each composed of two oligonucleotides including an overlapping sequence with each other, is present, the resulting multiple overlapping sequences are different in sequence from each other.

For example, in case of FIG. 2E, there are respective combinations between the forward primer oligonucleotide and pre-PC TO-1 (combination 1), between pre-PC TO-1 and pre-PC LO (combination 2), between pre-PC LO and pre-PC TO-2 (combination 3) and between pre-PC TO-2 and the reverse primer oligonucleotide (combination 4), with the overlapping sequences of combinations 1 to 4 being not identical or complementary to each other.

In an embodiment of the present disclosure, combinations, each consisting of two oligonucleotides including an overlapping sequence, selected from among the oligonucleotides included in the mixture, may have the same or different Tm values. For example, when among the oligonucleotides in the mixture is a plurality of combinations, each consisting of two oligonucleotides including an overlapping sequence with each other, Tm of overlapping sequence may differ from one combination to another or be identical for all the combinations, or may be different for some of the combinations and identical for the rest.

In an embodiment of the present disclosure, all the combinations, each consisting of two oligonucleotides including an overlapping sequence therebetween, selected from among the oligonucleotides in the mixture, may have the same Tm.

According to an embodiment, the pre-positive control oligonucleotides may be designed so that the overlapping sequence has a Tm in a specific temperature range depending on the annealing temperatures at which the target nucleic acid-detecting oligonucleotides hybridize with a target nucleic acid sequence.

In an embodiment of the present disclosure, Tm of the overlapping sequences in all the combinations of the oligonucleotides included in the mixture may be selected from the temperatures within a predetermined range of the Tm of the target nucleic acid-detecting oligonucleotides, for example, within a range of Tm±1° C., ±2° C., ±3° C., ±4° C., ±5° C., ±7° C., ±8° C., ±9° C., ±10° C., ±15° C., or ±20° C.

In an embodiment of the present disclosure, the overlapping sequence may have a Tm of 35° C. or higher, 40° C. or higher, 42° C. or higher, 45° C. or higher, 48° C. or higher, 50° C. or higher, 52° C. or higher, 55° C. or higher, or 57° C. or higher.

In an embodiment of the present disclosure, the overlapping sequence may have a Tm of 70° C. or less, 68° C. or less, 65° C. or less, 62° C. or less, or 60° C. or less.

In an embodiment of the present disclosure, the overlapping sequence may have a Tm of 35° C. to 70° C., 40° C. to 70° C., 45° C. to 70° C., 48° C. to 70° C., 50° C. to 70° C., 52° C. to 70° C., 55° C. to 70° C., 57° C. to 70° C., 35° C. to 68° C., 40° C. to 68° C., 45° C. to 68° C., 48° C. to 68° C., 50° C. to 68° C., 52° C. to 68° C., 55° C. to 68° C., 57° C. to 68° C., 35° C. to 65° C., 40° C. to 65° C., 45° C. to 65° C., 48° C. to 65° C., 50° C. to 65° C., 52° C. to 65° C., 55° C. to 65° C., 57° C. to 65° C., 45° C. to 68° C., 50° C. to 65° C., 52° C. to 62° C., or 55° C. to 60° C.

In an embodiment of the present disclosure, for a combination of two oligonucleotides including identical sequences to each other as an overlapping sequence, Tm of the overlapping sequence could be calculated as a Tm of the overlapping sequence between any one of the two oligonucleotides and a complementary sequence to the other oligonucleotide.

In an embodiment of the present disclosure, when the pre-positive control oligonucleotide included in the pre-positive control composition is a double-stranded form, a denaturation process of a double strand may be further conducted prior to the hybridization step.

A double strand may be denatured using conventional techniques including heating, treatment with alkali, formamide, urea, glycoxal, an enzymatic method (e.g., helicase), and binding protein, but with no limitations thereto. For example, the denaturation may be achieved by heating in a temperature range of 80-105° C. The single-strand formation of a target nucleic acid sequence can also be achieved by proteins used for isothermal amplification processes (e.g., LAMP, RPA, etc.). With respect to general methods for such processes, reference may be made to the document [Joseph Sambrook, et al., Molecular Cloning, A Laboratory Manual, Cold Spring Harbor Laboratory Press, Cold Spring Harbor, N.Y. (2001)].

(b-2) Extension of Oligonucleotide

Either or both of the two oligonucleotides hybridized with each other can be extended.

For example, in the case of FIG. 2E, the two complementary combinations, that is, (i) a forward primer oligonucleotide and a pre-positive control templating oligonucleotide (pre-PC TO-1) and (ii) a pre-positive control linking oligonucleotide (pre-PC LO) and a pre-positive control templating oligonucleotide (pre-PC TO-2) may be hybridized with each other via respective overlapping sequences and extended, with each serving as a template.

In an embodiment of the present disclosure, extension of the oligonucleotides may be achieved by a template-dependent extension reaction.

As used herein, the term “template-dependent extension reaction” refers to a reaction through which an oligonucleotide molecule hybridized with a target sequence is extended by sequentially adding nucleotides to the terminal moiety of the oligonucleotide molecule. In this regard, the extended sequence is determined by the complementary template sequence.

In an embodiment of the present disclosure, the extension of oligonucleotides may be performed by a template-dependent polymerase. For example, examples of the template-dependent polymerase include the “Klenow” fragment of E. coli DNA polymerase I, a thermostable DNA polymerase, and bacteriophage T7 DNA polymerase. In an embodiment of the present disclosure, the template-dependent polymerase is a thermostable DNA polymerase from at least one selected from various bacterial species. Concrete examples include thermostable DNA polymerases obtainable from Thermus aquaticus(Taq), Thermus thermophilus (Tth), Thermus filiformis, Thermis flavus, Thermococcus literalis, Thermus antranikianii, Thermus caldophilus, Thermus chliarophilus, Thermus flavus, Thermus igniterrae, Thermus lacteus, Thermus oshimai, Thermus ruber, Thermus rubens, Thermus scotoductus, Thermus silvanus, Thermus species Z05, Thermus species sps 17, Thermus thermophilus, Thermotoga maritima, Thermotoga neapolitana, Thermosipho africanus, Thermococcus litoralis, Thermococcus barossi, Thermococcus gorgonarius, Thermotoga maritima, Thermotoga neapolitana, Thermosipho africanus, Pyrococcus furiosus(Pfu), Pyrococcus woesei, Pyrococcus horikoshii, Pyrococcus abyssi, Pyrodictium occultum, Aquifex pyrophilus, and Aquifex aeolicus. In greater detail, the template-dependent DNA polymerase is Taq polymerase.

(b-3) Production of Complete-Positive Control Oligonucleotide

In an embodiment of the present disclosure, the product of the extension may be a complete-positive control oligonucleotide.

In another embodiment of the present disclosure, the product of the extension may be an intermediate-positive control oligonucleotide.

As used herein, the term “intermediate-positive control oligonucleotide” refers to an intermediate product resulting from the assembly of two oligonucleotides among one or more pre-positive control oligonucleotides or one or more pre-positive control oligonucleotides and one or more target nucleic acid-detecting oligonucleotides through an overlapping sequence therebetween. An intermediate-positive control oligonucleotide may be assembled with another oligonucleotide or another intermediate-positive control oligonucleotide to produce another intermediate-positive control oligonucleotide (e.g., a longer intermediate-positive control oligonucleotide) or a complete-positive control oligonucleotide. An intermediate-positive control oligonucleotide does not include the full sequence of the target nucleic acid-detecting oligonucleotide(s) and thus is distinguished from a complete-positive control oligonucleotide that includes the full sequence of the target nucleic acid-detecting oligonucleotide(s).

In an embodiment of the present disclosure, when an intermediate-positive control oligonucleotide for a target nucleic acid sequence is produced, at least one round of a step of denaturing a double-stranded nucleic acid and steps (b-1) and (b-2) may be further repeated to produce a complete-positive control oligonucleotide.

For instance, in the case of FIG. 2E, two intermediate-positive control oligonucleotides are produced by assembly of two complementary combinations as in FIG. 2E (b). The two intermediate-positive control oligonucleotides are dissociated into four single-stranded oligonucleotides through double-stranded nucleic acid denaturation. Of them, the two oligonucleotides including respective overlapping sequences complementary to each other are hybridized with each other and extend to finally produce a complete-positive control oligonucleotide, as shown in FIG. 2E (c).

According to an embodiment of the present disclosure, the number of intermediate-positive control oligonucleotides essentially required for producing a complete-positive control oligonucleotide may vary depending on sequences, forms (single- and double-stranded), directionality (in case of single-stranded form), and numbers of the pre-positive control oligonucleotides. The number of repeating rounds may vary depending on the number of the intermediate-positive control oligonucleotides necessarily required and/or whether overlapping sequences in combinations, each comprised of two oligonucleotides including an overlapping sequence, are identical or complementary.

Particularly, for the identical combination (e.g., forward primer and pre-PC TO in FIG. 2A), the two oligonucleotides cannot be directly hybridized to produce an intermediate-positive control oligonucleotide or a complete-positive control oligonucleotide. In this case, any one oligonucleotide (e.g., pre-PC TO in FIG. 2A) of the two oligonucleotides is used, together with another oligonucleotide (e.g., reverse primer in FIG. 2A) including an overlapping sequence complementary to the corresponding oligonucleotide, to produce intermediate-positive control oligonucleotides. Of the produced intermediate-positive control oligonucleotides, an oligonucleotide (e.g., an extended product of the reverse primer in FIG. 2A) including a complementary sequence to the identical overlapping sequence may be hybridized with the other oligonucleotide (e.g., forward primer in FIG. 2A) to produce a different intermediate-positive control oligonucleotide or a complete-positive control oligonucleotide.

In an embodiment of the present disclosure, the number of the repeating rounds may be one or more, two or more, three or more, four or more, five or more, six or more, seven or more, eight or more, nine or more, or ten or more.

In an embodiment of the present disclosure, when the round is repeated two or more times, the condition for the reaction (temperature, reaction times, etc.) may be the same for all of the rounds or differs from one round to another. For example, annealing temperature and/or reaction time for the first reaction round may be identical to or different from that for the second reaction round. The condition for the repeating round may be determined depending on the Tm of the overlapping sequence.

In an embodiment, a nucleic acid denaturation process may be conducted between the rounds.

In an embodiment of the present disclosure, the complete-positive control oligonucleotide is produced in a double-stranded form, any one strand of which comprises a sequence of the one or more pre-positive control oligonucleotides.

Step (c): Amplification of Complete-Positive Control Oligonucleotide

In the present disclosure, the complete-positive control oligonucleotide produced through step (b) is amplified using a forward and a reverse primer oligonucleotide for target nucleic acid amplification (see FIG. 2E (d)).

In an embodiment of the present disclosure, the amplification is conducted in a condition suitable for the amplification of a target nucleic acid sequence. In an embodiment of the present disclosure, the amplification may be conducted by polymerase chain reaction (PCR).

PCR for nucleic acid amplification is widely used in the field and includes cycles of denaturing a target nucleic acid molecule, annealing (hybridizing) to a target nucleic acid sequence, and extending a primer (U.S. Pat. Nos. 4,683,195, 4,683,202, and 4,800,159; Saiki et al., (1985) Science 230, 1350-1354).

Annealing of a primer to a target nucleic acid sequence may be conducted in a suitable hybridization condition determined by a general optimization procedure. Conditions such as temperatures, concentrations of ingredients, hybridization, numbers of washing, buffer ingredients, pH and ion strength thereof, and so on may be determined depending on various factors including sequence lengths and GC contents of oligonucleotides (primers) and target nucleotide sequences. For detailed conditions for hybridization, reference may be made to Joseph Sambrook et. al., Molecular Cloning, A Laboratory Manual, Cold Spring Harbor Laboratory Press, Cold Spring Harbor, N.Y. (2001); and M. L. M. Anderson, Nucleic Acid Hybridization, Springer-Verlag New York Inc. N.Y. (1999).

A primer annealed to a target sequence can be extended by a template-dependent polymerase.

When a polymerization reaction starts, components necessary therefor may be supplied in excess in a reaction container. In the context of components for extension, an excessive amount means a sufficient amount of each component such that the ability to achieve the desired extension is not substantially limited by the concentrations of the components. In order to achieve a desired degree of extension, cofactors such as Mg²⁺, etc., dATP, dCTP, dGTP, and dTTP are preferably supplied in sufficient quantity to a reaction mixture.

According to another embodiment, ligase chain reaction (LCR, see Wiedmann M, et al., “Ligase chain reaction (LCR)-overview and applications.” PCR Methods and Applications 1994 February; 3(4):S51-64), gap filling LCR (GLCR, see WO 90/01069, European Patent Number 439182, and WO 93/00447), Q-beta replicase amplification (Q-beta, see Cahill P, et al., Clin Chem., 37(9): 1482-5(1991), and U.S. Pat. No. 5,556,751), strand displacement amplification (SDA, see G T Walker et al., Nucleic Acids Res. 20(7):16911696(1992), and European Patent Number 497272), nucleic acid sequence-based amplification (NASBA, see Compton, J. Nature 350(6313): 912(1991)), transcription-mediated amplification (TMA, see Hofmann W P et al, J Clin Virol. 32(4):289-93(2005); U.S. Pat. No. 5,888,779), rolling circle amplification (RCA, see Hutchison C. A. et al., Proc. Natl Acad. Sci. USA. 102:1733217336(2005)), recombinase polymerase amplification (RPA), and loop-mediated isothermal amplification (LAMP) may be utilized as a method for amplifying a target nucleic acid sequence, but with no limitations thereto.

In an embodiment of the present disclosure, the pre-positive control composition may be adopted to carry out positive control reactions for a plurality of target nucleic acid-detecting compositions.

In an embodiment of the present disclosure, the plurality of target nucleic acid-detecting compositions may include a plurality of oligonucleotides for detecting a plurality of target nucleic acids (e.g., a plurality of primers for amplifying a plurality of target nucleic acids), and the pre-positive control composition may include a combination of pre-positive control oligonucleotides from which complete-positive control oligonucleotides for individual target nucleic acids can be produced during a reaction process. In this regard, the number of the pre-positive control oligonucleotides included so as to produce complete-positive control oligonucleotides for individual target nucleic acids may be identical or different according to the individual target nucleic acids.

According to an embodiment of the present disclosure, the method of the present disclosure may further comprise a step of detecting an amplicon of the complete-positive control oligonucleotide produced by the method of the present disclosure.

The target nucleic acid-detecting composition may comprise an ingredient (e.g., labeled oligonucleotide) capable of providing a signal detectable depending on the existence of a target nucleic acid sequence, and a complete-positive control oligonucleotide can be detected using the ingredient.

According to another embodiment of the present disclosure, a post-PCR detection method or a real-time detection method is used as a method for detecting an amplicon of the complete-positive control oligonucleotide produced according to the method of the present disclosure.

The real-time detection method may be conducted using a non-specific fluorescent dye which can be non-specifically intercalated into a duplex, which is an amplicon of a target nucleic acid sequence.

In addition, the real-time detection method may utilize a labeled probe capable of specifically hybridizing with a target nucleic acid sequence. Examples of the method include a molecular beacon method using a dual-labeled probe capable of forming a hairpin structure (Tyagi et al, Nature Biotechnology v.14 Mar. 1996), a hybridization probe method using two probes singly labeled with a donor or acceptor (Bernad et al, 147-148 Clin Chem 2000; 46), a Lux method using a single-labeled oligonucleotide (U.S. Pat. No. 7,537,886), and a TaqMan method using a cleavage reaction of a dual-labeled probe by the 5′-nuclease activity of DNA polymerase as well as hybridization of a dual-labeled probe (U.S. Pat. Nos. 5,210,015 and 5,538,848), but are not limited thereto.

In addition, real-time detection may be carried out using a duplex formed depending on the existence of a target nucleic acid sequence. The duplex formed depending on the existence of a target nucleic acid sequence is not the target sequence amplicon itself formed through an amplification reaction, but a duplex the amount of which increases in proportion to the amplification of the target nucleic acid sequence. The duplex formed depending on the existence of a target nucleic acid sequence can be obtained according various methods, for example, the PTO cleavage and extension (PTOCE) method disclosed in WO 2012/096523 the content of which is incorporated herein by reference.

Furthermore, the real-time detection of a target may be achieved by a method for detecting at least one target nucleic acid sequence wherein signals detected at different temperatures are used with a single-type of label, as disclosed in WO 2015/147370, WO 2015/147377, WO 2015/147382, and WO 2015/147412, the contents of which are incorporated herein by reference.

The post-PCR detection method is a method for detecting an amplicon after nucleic acid amplification. Examples of the post-PCR detection method include separation of amplicons by size (generally, conducted by gel electrophoresis) and separation of amplicons through fixation, but are not limited thereto.

In addition, available as the post-PCR detection method is a post-PCR melting assay in which fluorescent intensities are monitored while the temperature is increased or decreased within a predetermined range after amplification of a target nucleic acid sequence, and then amplicons are detected using a melting profile (U.S. Pat. Nos. 5,871,908 and 6,174,670 and WO 2012/096523).

Unless otherwise defined, all technical and scientific terms used herein have the same meaning as commonly understood by one of ordinary skill in the art.

A different aspect of the present disclosure provides a pre-positive control composition comprising a pre-positive control oligonucleotide:
wherein the pre-positive control oligonucleotide comprises a sequence overlapping with at least one oligonucleotide selected from among target nucleic acid-detecting oligonucleotides included in a target nucleic acid-detecting composition and a different pre-positive control oligonucleotide, wherein the overlapping sequence of the pre-positive control oligonucleotide is identical or complementary to an overlapping sequence of the at least one oligonucleotide;
wherein the pre-positive control composition comprises one or more pre-positive control oligonucleotides;
wherein the one or more pre-positive control oligonucleotides are selected so as to produce the complete-positive control oligonucleotide by assembling (i) the pre-positive control oligonucleotides or (ii) the one or more pre-positive control oligonucleotides and the at least one of the target nucleic acid-detecting oligonucleotides;
wherein a mixture of the pre-positive composition and the target nucleic acid-detecting composition comprises combinations of two oligonucleotides comprising an overlapping sequence identical or complementary to each other; and
whereby the pre-positive control composition is capable of producing a complete-positive control oligonucleotide for a positive control reaction for the target nucleic acid-detecting composition.

Since “a pre-positive control composition comprising a pre-positive control oligonucleotide” of the present disclosure is to carry out the above-described method of the present disclosure, the common descriptions therebetween are omitted in order to avoid undue redundancy leading to the complexity of this specification.

In an embodiment of the present disclosure, the one or more pre-positive control oligonucleotides may include a sequence overlapping with at least one of the target nucleic acid-detecting oligonucleotides.

In an embodiment of the present disclosure, the target nucleic acid-detecting oligonucleotides may include a forward primer oligonucleotide and a reverse primer oligonucleotide for target nucleic acid amplification.

In an embodiment of the present disclosure, the one or more pre-positive control oligonucleotide may include a sequence overlapping with at least one of the forward primer oligonucleotide and the reverse primer oligonucleotide for target nucleic acid amplification.

In an embodiment of the present disclosure, the pre-positive control oligonucleotide may include a partial sequence of the complete-positive control oligonucleotide for the target nucleic acid sequence.

In an embodiment of the present disclosure, the complete-positive control oligonucleotide formed using the composition of the present disclosure is in a double-stranded form, any one strand of which may include a sequence of the one or more pre-positive control oligonucleotides.

In an embodiment of the present disclosure, the 3′-end of the pre-positive control oligonucleotide is extendable therefrom or blocked to prevent extension therefrom.

In an embodiment of the present disclosure, the combinations of two oligonucleotides including an overlapping sequence identical or complementary to each other in the mixture of the pre-positive composition and the target nucleic acid-detecting composition may have overlapping sequences being neither identical nor complementary to each other.

In an embodiment of the present disclosure, the pre-positive control oligonucleotide may be a pre-positive control templating oligonucleotide or a pre-positive control linking oligonucleotide.

In an embodiment of the present disclosure, the pre-positive control templating oligonucleotide may include a sequence that overlaps with at least one of the forward primer oligonucleotide or reverse primer oligonucleotide and may not include sequences that overlap totally with the full sequences of the forward primer oligonucleotide and the reverse primer oligonucleotide.

In an embodiment of the present disclosure, the pre-positive control linking oligonucleotide may include sequences overlapping with two different pre-positive control oligonucleotides at its 3′-end and 5′-end and may not include a sequence overlapping with the forward primer oligonucleotide or the reverse primer oligonucleotide.

In an embodiment of the present disclosure, the pre-positive control composition may include one to five pre-positive control oligonucleotides.

In an embodiment of the present disclosure, the pre-positive control composition may include one pre-positive control oligonucleotide wherein the one pre-positive control oligonucleotide may be one pre-positive control templating oligonucleotide.

In an embodiment of the present disclosure, the pre-positive control composition may include one pre-positive control oligonucleotide wherein the one pre-positive control oligonucleotide may include one pre-positive control templating oligonucleotide. The one pre-positive control templating oligonucleotide may include sequences overlapping with the forward primer oligonucleotide and reverse primer oligonucleotide at its 3′-end and 5′-end and may not include sequences overlapping totally with the full sequences of the forward primer oligonucleotide and the reverse primer oligonucleotide.

In an embodiment of the present disclosure, the pre-positive control composition may include two pre-positive control oligonucleotides wherein the two pre-positive control oligonucleotides may include two pre-positive control templating oligonucleotides.

In an embodiment of the present disclosure, the pre-positive control composition may include three pre-positive control oligonucleotides wherein the three pre-positive control oligonucleotides may include two pre-positive control templating oligonucleotides and one pre-positive control linking oligonucleotide.

In an embodiment of the present disclosure, the pre-positive control composition may include four pre-positive control oligonucleotides wherein the four pre-positive control oligonucleotides may include two pre-positive control templating oligonucleotides and two pre-positive control linking oligonucleotides.

In an embodiment, when the pre-positive control composition comprises two or more pre-positive control oligonucleotides, the two or more pre-positive control oligonucleotides includes two pre-positive control templating oligonucleotide, wherein the two pre-positive control templating oligonucleotides may include a first pre-positive control templating oligonucleotide including a sequence overlapping with the forward primer oligonucleotide and a second pre-positive control templating oligonucleotide including a sequence overlapping with the reverse primer oligonucleotide.

In an embodiment of the present disclosure, wherein the target nucleic acid-detecting oligonucleotides may further include a probe.

The pre-positive control composition according to the present disclosure may further include a buffer.

The buffer may be a TE buffer.

In an embodiment, the pre-positive control composition is designed to conduct a positive control reaction for a plurality of target nucleic acids-detecting composition.

Provided according to another aspect of the present disclosure is a kit for conducting a positive control reaction for a target nucleic acid-detecting composition, the kit comprising:
(a) a target nucleic acid-detecting composition, wherein the target nucleic acid-detecting composition includes target nucleic acid-detecting oligonucleotides comprising a forward primer oligonucleotide and a reverse primer oligonucleotide for target nucleic acid amplification; and
(b) a pre-positive control composition comprising a pre-positive control oligonucleotide,
wherein the pre-positive control oligonucleotide comprises a sequence overlapping with at least one oligonucleotide selected from among target nucleic acid-detecting oligonucleotides included in a target nucleic acid-detecting composition and a different pre-positive control oligonucleotide, wherein the overlapping sequence of the pre-positive control oligonucleotide is identical or complementary to an overlapping sequence of the at least one oligonucleotide;
wherein the pre-positive control composition comprises one or more pre-positive control oligonucleotides;
wherein the one or more pre-positive control oligonucleotides are selected so as to produce the complete-positive control oligonucleotide by assembling (i) the pre-positive control oligonucleotides or (ii) the one or more pre-positive control oligonucleotides and the at least one of the target nucleic acid-detecting oligonucleotides;
wherein a mixture of the pre-positive composition and the target nucleic acid-detecting composition comprises combinations of two oligonucleotides comprising an overlapping sequence identical or complementary to each other; and
whereby the pre-positive control composition is capable of producing a complete-positive control oligonucleotide for a positive control reaction for the target nucleic acid-detecting composition.

Since the “kit for conducting a positive control reaction for a target nucleic acid-detecting composition” of the present disclosure is to carry out the above-described method of the present disclosure, the common descriptions therebetween are omitted in order to avoid undue redundancy leading to the complexity of this specification.

### Advantageous Effects of Invention

Features and advantages of the present disclosure are summarized as follows:
(a) The pre-positive control composition of the present disclosure is not provided as a complete positive control, but as one or more pre-positive control oligonucleotides including partial sequences of a complete-positive control oligonucleotide until an experiment preparation step for a positive control reaction, wherein the complete-positive control oligonucleotide is produced through a positive control reaction leading to assembly between a plurality of oligonucleotides, for example, assembly between two or more pre-positive control oligonucleotides or between one or more pre-positive control oligonucleotides and one or more target nucleic acid-detecting oligonucleotides.
(b) The sequence of a complete-positive control produced by a positive control reaction using a pre-positive control composition can be desirably designed by selecting various combinations of the sequence and number of the pre-positive control oligonucleotide.
(c) Conventional positive controls suffer from the problem that contamination is generated by positive controls from positive control preparation to an experiment preparation step for a positive control reaction. In contrast, because a complete-positive control is produced from the pre-positive control composition through a positive control reaction, it is possible to minimize the contamination problem of the positive control during the preparation of the pre-positive control composition and in an experiment preparation step for a positive control reaction.
(d) When contamination occurs with a complete-positive control, the contamination source can be discriminated because the complete-positive control is designed to have a sequence different from an amplicon of a target nucleic acid.
(e) A Ct value change rate according to 10-fold diluted concentrations of the pre-positive control composition is larger than that according to 10-fold diluted concentrations of conventional plasmid DNA positive controls, implying that it is easier to control contamination with the pre-positive control composition than conventional plasmid DNA positive controls.
(f) The pre-positive control composition, which is in an oligonucleotide form, enjoys the stability that oligonucleotides retain and as such, can be conveniently stored.

### BRIEF DESCRIPTION OF DRAWINGS

FIGS. 1A-1D are schematic views showing various examples of pre-positive control compositions including 1 to 5 pre-positive control oligonucleotides used in a positive control reaction for a pair of primers for target nucleic acid amplification and a probe for target nucleic acid detection. In the figures, the arrows indicate 5′-3′ direction. The forward primer and the reverse primer for target detection are discriminated by the directions of the arrows.

FIGS. 2A-2E are schematic views illustrating examples of positive control reactions using pre-positive control compositions including 1 to 3 pre-positive control oligonucleotides.

FIG. 3 shows Ct values of positive control reactions using pre-PC compositions.

FIG. 4 shows Ct values of positive control reactions using comparative positive controls.

FIG. 5 shows Ct value change rates according to concentrations of pre-PC compositions.

FIG. 6 shows Ct value change rates according to concentrations of a comparative positive control composition.

FIG. 7 shows Ct values of positive control reactions using pre-PC compositions or a comparative positive control composition for the target nucleic acid-detecting compositions of Table 8.

FIG. 8 shows Ct values of multiplex positive control reactions using multiplex pre-PC compositions.

FIG. 9 shows Ct values of multiplex positive control reactions using multiplex comparative positive control compositions.

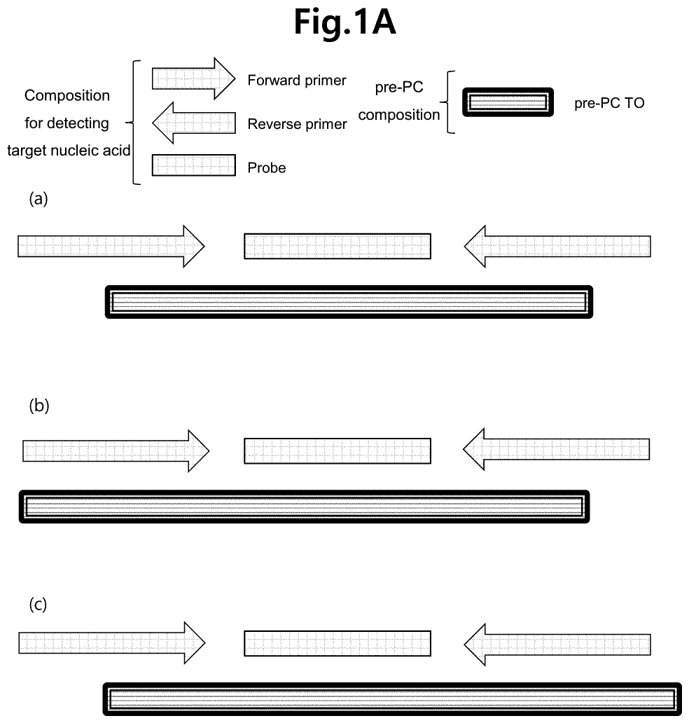

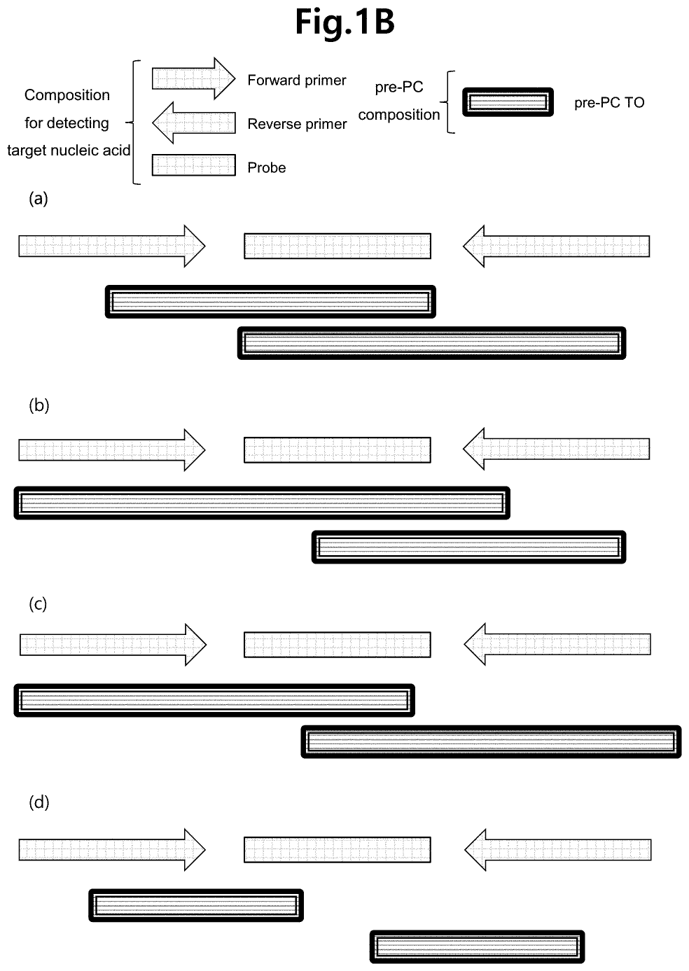

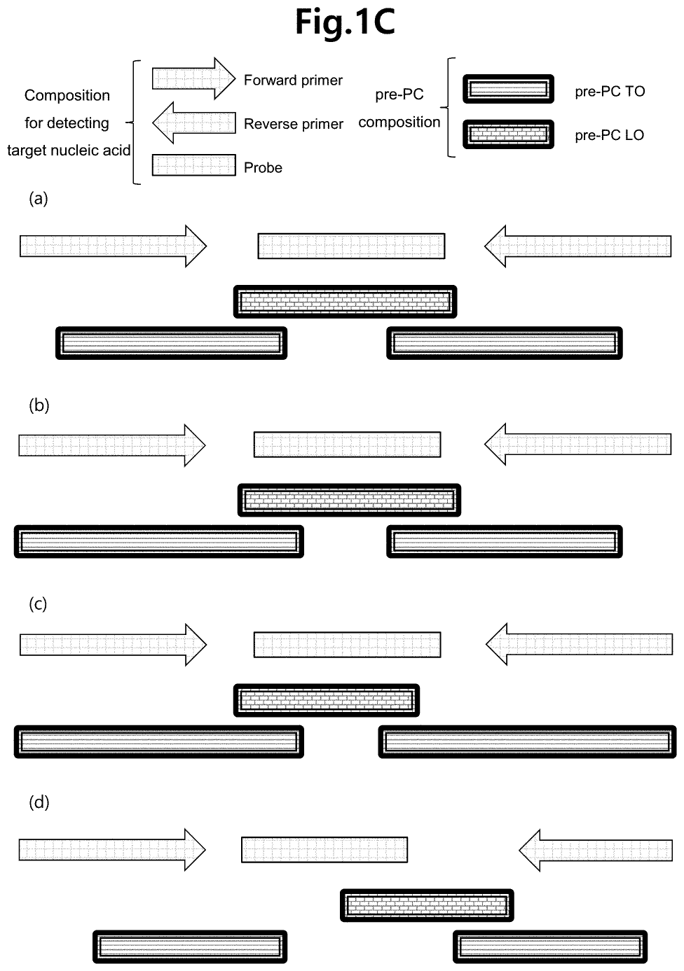

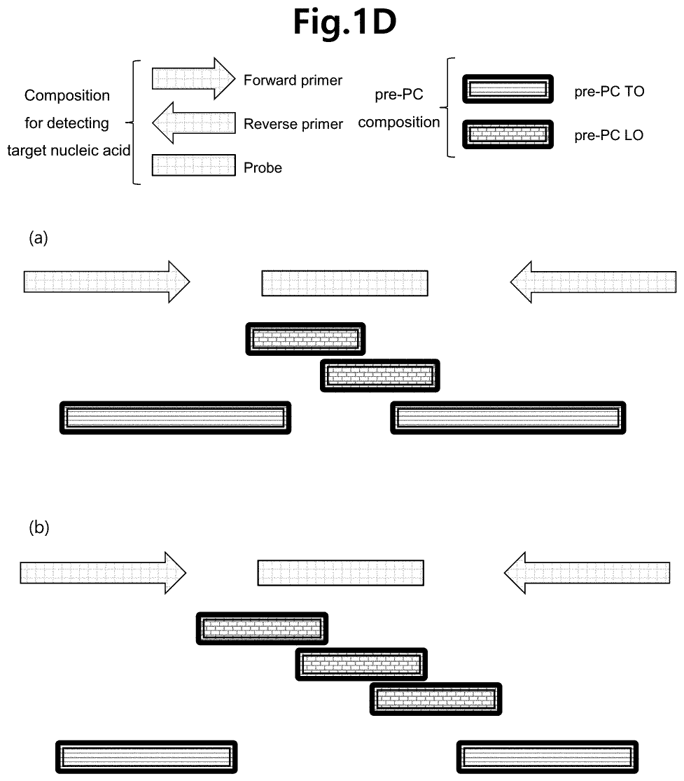

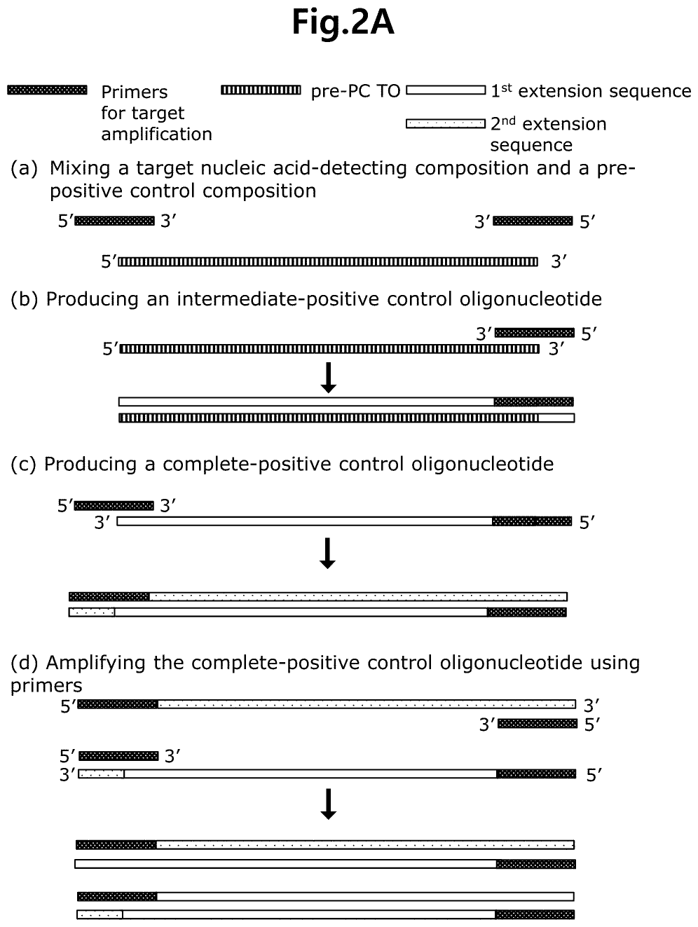

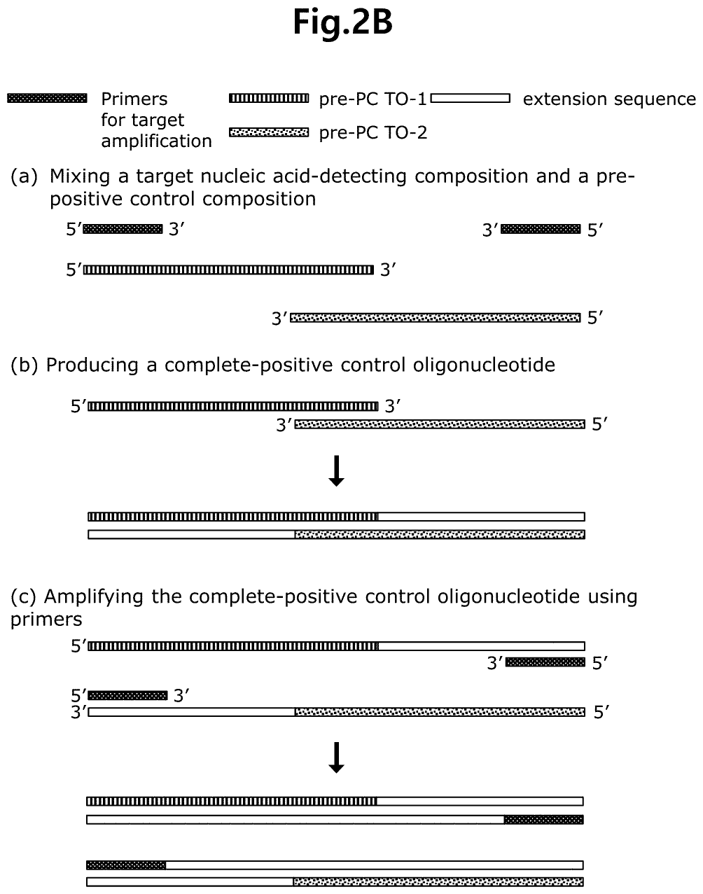

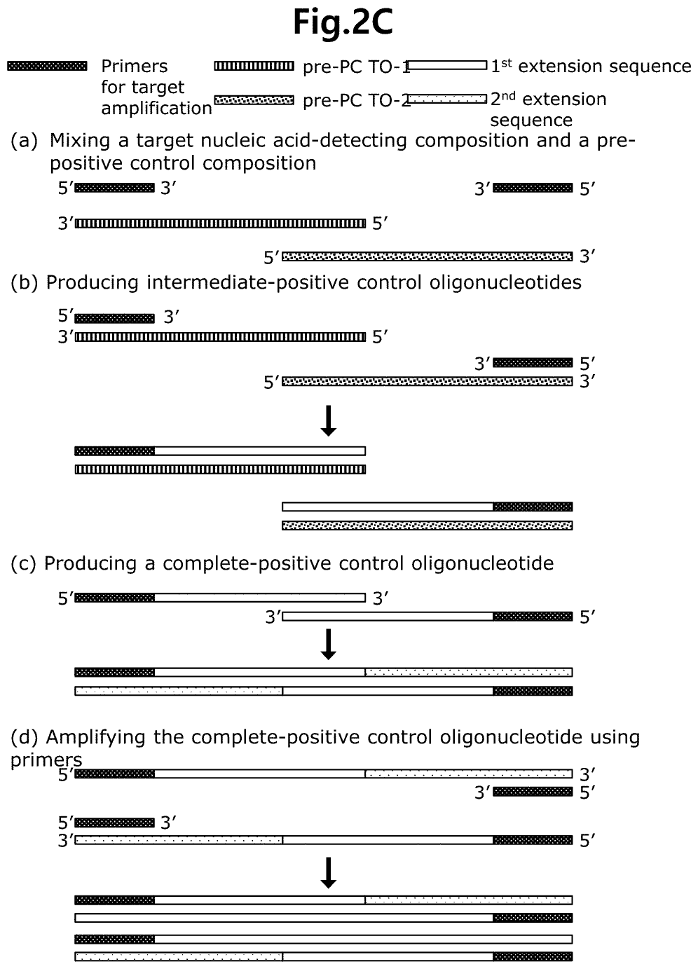

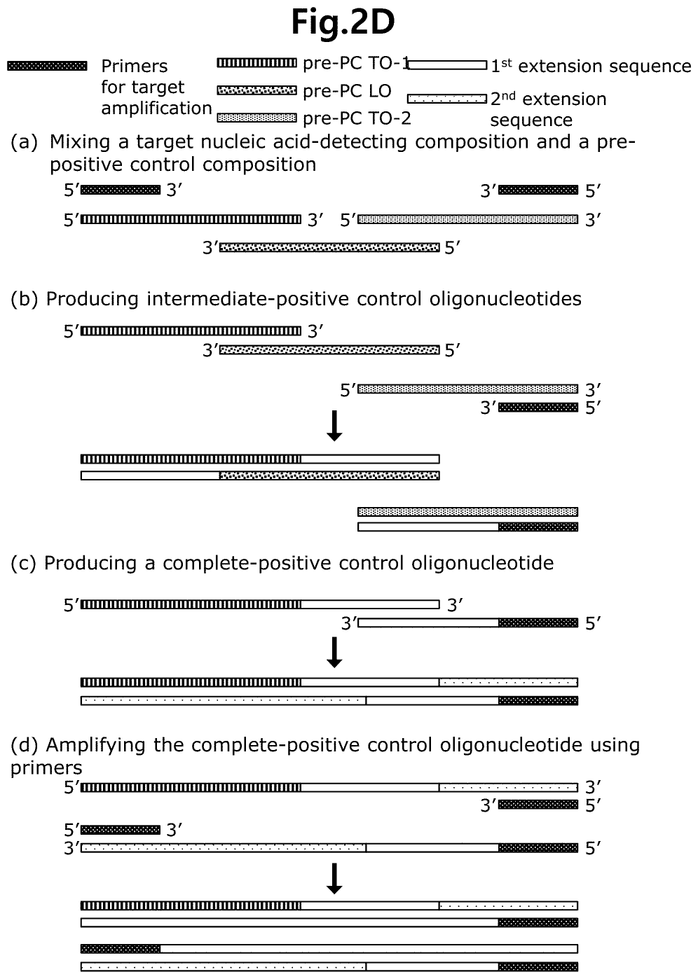

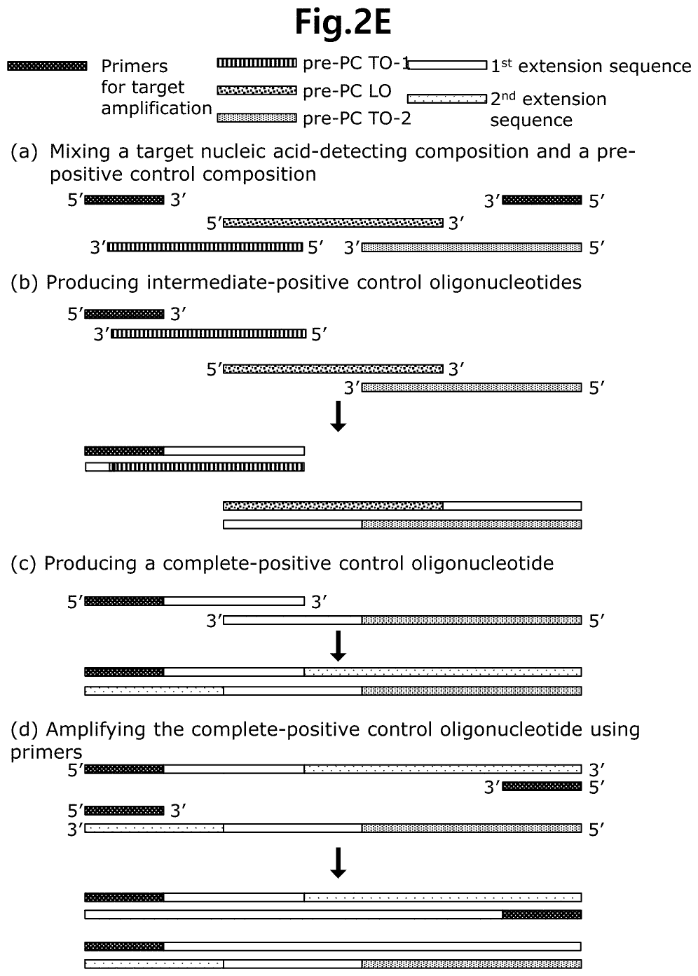

### BEST MODE FOR CARRYING OUT THE INVENTION

The present disclosure will now be described in further detail by examples. It would be obvious to those skilled in the art that these examples are intended to be more concretely illustrative and the scope of the present disclosure as set forth in the appended claims is not limited to or by the examples.

### EXAMPLES

Assays were made of workability of the pre-positive control composition (hereinafter referred to as “pre-PC composition”) of the present disclosure in implementing a positive control reaction and availability of the pre-PC composition as a positive control in evaluating the operability of target nucleic acid-detecting oligonucleotides. In addition, the positive control reaction according to the present disclosure was assayed in comparison with conventional plasmid DNA positive control.

### Example 1: Preparation of Composition

### (1-1) Preparation of Target Nucleic Acid-Detecting Composition

Compositions for use in detecting target nucleic acid sequences by the TaqMan real-time PCR method (U.S. Pat. Nos. 5,210,015 and 5,538,848) was prepared.

As target nucleic acids, NG gene (see: AccID: AP023075.1), CT gene (see: AccID: CP016427.1), HBB gene (see: AccID: GU324922.1), and PSX gene (see: AccID: NM 008955.1) were used, the sequences of which are given as listed in Table 1, below.

TABLE 1
Target Nucleic Acid Sequence

| Seq | | | |
|---|---|---|---|
| ID | | | |
| No. | Target | Type | Sequence (5′ to 3′) |
| 1 | NG | Amplicon | CGAAATTGCCGCCACTGCTTCCTACCGCTTCGGTAATACAGTCCC |
| | | | GCGCATCAGCTATGCCCATGGTTTCGACTTTGTCGAACGCAGTCA |
| | | | GAAACGCGAACATACCAGCTATGATCAAATCATCGCCGGTGTCGA |
| | | | TTACGATTTTTCCAAGCGCACTTCCGCCATCATGTCTGCCGCTTG |
| | | | GCTGAAACGA |
| 2 | CT | Amplicon | CTTTTTCCGCATCCAAACCAATTGTAATAGAAGCATTGGTTGATG |
| | | | AATTATTGGAGACTGTTAAAGATATTCCATCAGAAGCTGTCATTT |
| | | | TGGCTGCGACAGGTGTTGATGTTGTCCCAAGGATTATTTGCTGGT |
| | | | CCTTGAGCGGCTCTGTCATTTGCCCAACTTTGATATTATCAGCAA |
| | | | AGACGCAGTTTTCAGTGTTATACAAATAAAAACCAGAATTTCCCA |
| | | | TTTTAAAACTCTTTTTTATTTTGAGCTTTAA |
| 3 | HBB | Amplicon | CCTTCAGTTAGCTTTGATCTTGTTTATTTCTTGTCTTCTGCTAGA |
| | | | TTTAGGGTTGGTTTGCTCTTGGTTCTCTGGTTCTTTTAGTTGTGA |
| | | | CATTAGGTTGTTAATTTGAGGGCTTTAAGACTTTTTGATGTGGGC |
| | | | ATTTAGTGTATAAATTTCTCTCTTAACACTGTCTAAGCTGTGTCC |
| | | | CAGAGATTCCG |
| 4 | PSX | Amplicon | AGCCAGTCCTGATAGCATCAGAAACCCACATGTTCTGAATAGGCT |
| | | | GGCTCAACTGCGGTACAGACGCACCAGGTTCACCCACTCTCAGCT |
| | | | GCATGACCTGGAGCGCCTTTTCCAAGAGACTCGCTACCCCAGCTT |
| | | | GCGAGCAAGGAGGGATCTTGCACGATGGATGGGTGTGGATGAATG |
| | | | TGATGTGCAGAA |

(a) Preparation of NG Target Nucleic Acid-Detecting Composition

For use in amplifying and detecting a NG target nucleic acid used in this Example, sequences of a forward primer oligonucleotide, a reverse primer oligonucleotide, and a probe oligonucleotide are given as listed in Table 2, below.

TABLE 2
NG Target Nucleic Acid-Detecting Oligonucleotides

| Seq | | | |
|---|---|---|---|
| ID | | | |
| No. | Target | Type | Sequence (5′ to 3′) |
| 5 | NG | F primer | CGAAATTGCCGCCACTGIIIIITACCGCTTC |
| 6 | NG | R primer | TCGTTTCAGCCAAGCGGIIIIIATGATGGCG |
| 7 | NG | probe | [FAM]CGCGTTTCTGACTGCGTTCGAC |
| | | | [BHQ1] |

Extension of the forward primer oligonucleotide and the reverse primer oligonucleotide and cleavage of the TaqMan probe oligonucleotide were performed by Taq DNA polymerase having 5′ nuclease activity. The probe had a fluorescent reporter molecule labeled at the 5′-end thereof and a quencher molecule labeled at the 3′-end thereof.

(b) Preparation of Multiplex Target Nucleic Acid-Detecting Composition

Sequences of forward primer oligonucleotides, reverse primer oligonucleotides, and probe oligonucleotides for the four target nucleic acids used in this Example are given as listed in Table 3, below.

TABLE 3
Target Nucleic Acid-Detecting Oligonucleotide
by Target Nucleic Acid

| Seq | | | |
|---|---|---|---|
| ID | | | |
| No. | Target | Type | Sequence (5′ to 3′ ) |
| 5 | NG | F primer | CGAAATTGCCGCCACTGIIIIITACCGCTTC |
| 6 | NG | R primer | TCGTTTCAGCCAAGCGGIIIIIATGATGGCG |
| 7 | NG | probe | [FAM]CGCGTTTCTGACTGCGTTCGAC[BHQ1] |
| 8 | CT | F primer | CTTTTTCCGCATCCAAACCAATTIIIIIAGAAGCA |
| | | | TTG |
| 9 | CT | R primer | TTAAAGCTCAAAATAAAAAAGAGTTTIIIIITGGG |
| | | | AAATTC |
| 10 | CT | probe | [Cal Fluor Red 610] |
| | | | CCAACTTTGATATTATCAGCAAAGACGCAGTTTT |
| | | | [BHQ-2] |
| 11 | HBB | F primer | CCTTCAGTTAGCTTTGATCTTGTTIIIIICTTGTC |
| | | | TT |
| 12 | HBB | R primer | CGGAATCTCTGGGACACAGCIIIIICAGTGTTA |
| 13 | HBB | probe | [Cal Fluor Orange 560] |
| | | | GAGGGCTTTAAGACTTTTTGATGTGGGC [BHQ-1] |
| 14 | PSX | F primer | AGCCAGTCCTGATAGCATCIIIIICCCACATGTT |
| 15 | PSX | R primer | TTCTGCACATCACATTCATCIIIIICCATCCATCG |
| 16 | PSX | probe | [Quasar 670] |
| | | | TCGGGCACCAGGTTCACCCACTCTCAGGGCG |
| | | | [BHQ1-2] |

Extension of the forward primer oligonucleotides and the reverse primer oligonucleotides and cleavage of the TaqMan probe oligonucleotides were performed by Taq DNA polymerase having 5′ nuclease activity. Each of the probes had a fluorescent reporter molecule labeled at the 5′-end thereof and a quencher molecule labeled at the 3′-end thereof.

(1-2) Preparation of Pre-PC Composition

(a) Preparation of Pre-PC Composition for NG Target Nucleic Acid

For the NG target nucleic acid, pre-PC compositions including 1 to 4 pre-positive control oligonucleotides (hereinafter referred to as “pre-PC oligonucleotide”) were prepared at various concentrations.

The sequence of the complete-positive control produced using the pre-PC compositions is given in Table 4, below.

TABLE 4
Complete-Positive Control Oligonucleotide
Produced Using NG pre-PC Composition

| Seq | | | |
|---|---|---|---|
| ID | | | |
| No. | Target | TYPE | Sequence (5′ to 3′) |
| 17 | NG | Complete- | TCGTTTCAGCCAAGCGGGGGGGATGATGGCGT |
| | | positive | GTTCGCGTTTCTGACTGCGTTCGACTACCGAA |
| | | control | GCGGTACCCCCCAGTGGCGGCAATTTCG |
| | | oligonucleotide | |

(i) Preparation of NG pre-PC composition including one pre-PC oligonucleotide (hereinafter referred to as “NG pre-PC composition (i)”):

The one pre-PC oligonucleotide is one pre-positive control templating oligonucleotide (hereinafter referred to as “pre-PC TO”) and has the sequence shown in Table 5.

The pre-PC TO (SEQ ID NO: 18) was serially 2-fold diluted with a TE buffer to prepare NG pre-PC compositions (i) having five concentrations in total (concentration 1: 9.4×10⁶ copies/μl, concentration 2: 4.7×10⁶ copies/μl, concentration 3: 2.4×10⁶ copies/μl, concentration 4: 1.2×10⁶ copies/μl, and concentration 5: 5.9×10⁵ copies/μl).

(ii) Preparation of pre-PC composition including two pre-PC oligonucleotides (hereinafter referred to as “NG pre-PC composition (ii)”):

The two pre-PC oligonucleotides are two pre-PC TOs and have the sequences given in Table 5.

A pre-PC composition including pre-PC TO-1 (SEQ ID NO: 19) and pre-PC TO-2 (SEQ ID NO: 20) each at a concentration of 6.0×10⁸ copies/μl was serially 2-fold diluted with a TE buffer to prepare NG pre-PC compositions (ii) having five concentrations in total (concentration 1: 6.0×10⁸ copies/μl, concentration 2: 3.0×10⁸ copies/μl, concentration 3: 1.5×10⁸ copies/μl, concentration 4; 7.5×10⁷ copies/μl, and concentration 5: 3.8×10⁷ copies/μl).

(iii) Preparation of Pre-PC Composition Including Three Pre-PC Oligonucleotide (Hereinafter Referred to as “NG Pre-PC Composition (iii)”):

The three pre-PC oligonucleotides are two pre-PC TOs and one pre-positive control linking oligonucleotide (hereinafter referred to as “pre-PC LO”) and have the sequences given in Table 5.

A pre-PC composition including pre-PC TO-1 (SEQ ID NO: 21), pre-PC TO-2 (SEQ ID NO: 22), and pre-PC LO-1 (SEQ ID NO: 23) each at a concentration of 2.4×10⁹ copies/μl was serially 2-fold diluted with a TE buffer to prepare NG pre-PC compositions (iii) having five concentrations in total (concentration 1: 2.4×10⁹ copies/μl, concentration 2: 1.2×10⁹ copies/μl, concentration 3: 6.0×10⁸ copies/μl, concentration 4: 3.0×10⁸ copies/μl, and concentration 5: 1.5×10⁸ copies/μl).

(iv) Preparation of Pre-PC Composition Including Four Pre-PC Oligonucleotides (Hereinafter Referred to as “NG Pre-PC Composition (iv)”):

The four pre-PC oligonucleotides are two pre-PC TOs and two pre-PC LOs and have the sequences given in Table 5.

A pre-PC composition including pre-PC TO-1 (SEQ ID NO: 24), pre-PC TO-2 (SEQ ID NO: 25), pre-PC LO-1 (SEQ ID NO: 26), and pre-PC LO-2 (SEQ ID NO: 27) each at a concentration of 1.5×10¹¹ copies/μl was serially 2-fold diluted with a TE buffer to prepare NG pre-PC compositions (iv) having three concentrations in total (concentration 1: 1.5×10¹¹ copies/μl, concentration 2: 7.7×10¹⁰ copies/μl, and concentration 3: 3.9×10¹⁰ copies/μl).

TABLE 5
pre-PC Oligonucleotide for NG Target Nucleic Acid

| Seq | pre-PC | pre-PC | |
|---|---|---|---|
| ID | composition | oligonucleotide | |
| No. | TYPE | TYPE | Sequence (5′ to 3′) |
| 18 | NG pre-PC | pre-PC TO | GGGGGATGATGGCGTGTTCGCGTTTCTGACTGC |
| | composition | | GTTCGACTACCGAAGCGGTACCCCC |
| | (i) | | |
| 19 | NG pre-PC | pre-PC TO-1 | CGAAATTGCCGCCACTGCTTCCTACCGCTTCGG |
| | composition | | TAGTCGAACGCAGTCAGAAACG |
| 20 | (ii) | pre-PC TO-2 | TCGTTTCAGCCAAGCGGCAGACATGATGGCGTG |
| | | | TTCGCGTTTCTGACTGCGTTC |
| 21 | NG pre-PC | pre-PC TO-1 | CTGCGTTCGACTACCGAAGCGGTAGGAAGCAGT |
| | composition | | GGCGGCAATTTCG |
| 22 | (iii) | pre-PC TO-2 | TCGTTTCAGCCAAGCGGCAGACATGATGGCGTG |
| | | | TTCGCGTTTCTGA |
| 23 | | pre-PC LO-1 | CGGTAGTCGAACGCAGTCAGAAACGCGAACACG |
| 24 | NG pre-PC | pre-PC TO-1 | AAGCGGTAGGAAGCAGTGGCGGCAATTTCG |
| 25 | composition | pre-PC TO-2 | ACGCCATCATGTCTGCCGCTTGGCTGAAACGA |
| 26 | (iv) | pre-PC LO-1 | ACTGCTTCCTACCGCTTCGGTAGTCGAACGCAG |
| | | | TCAGAAACG |
| 27 | | pre-PC LO-2 | GGCAGACATGATGGCGTGTTCGCGTTTCTGACT |
| | | | GCGTTC |

(b) Preparation of Multiplex Pre-PC Composition for Multiple Target Nucleic Acids

An examination was made to see whether the pre-PC compositions according to the present disclosure could be used to conduct a positive control reaction for multiple target nucleic acids.

To this end, a multiplex pre-PC composition including two or three pre-PC oligonucleotides for each target nucleic acid was prepared for multiple target nucleic acids.

Sequences of the complete-positive control oligonucleotides produced using the multiplex pre-PC composition for multiple target nucleic acids are given as listed in Table 6, below.

TABLE 6
Complete-Positive Control Oligonucleotide
Produced Using Multiplex pre-PC Composition

| Seq | | | |
|---|---|---|---|
| ID | | | |
| No. | Target | TYPE | Sequence (5′ to 3′) |
| 17 | NG | complete- | TCGTTTCAGCCAAGCGGGGGGGATGATGGCGTGTTCGCGTTTCTG |
| | | positive | ACTGCGTTCGACTACCGAAGCGGTACCCCCCAGTGGCGGCAATTT |
| | | control | CG |
| 28 | CT | oligonucleotide | CTTTTTCCGCATCCAAACCAATTGGGGGAGAAGCATTGGTTGCCA |
| 29 | HBB | | ACTTTGATATTATCAGCAAAGACGCAGTTTTCAGTGAATTTCCCA |
| | | | CCCCCAAACTCTTTTTTATTTTGAGCTTTAA |
| | | | CGGAATCTCTGGGACACAGCGGGGGCAGTGTTAAGAGAGCCCACA |
| | | | TCAAAAAGTCTTAAAGCCCTCCTAGCAGAAGACAAGCCCCCAACA |
| | | | AGATCAAAGCTAACTGAAGG |
| 30 | PSX | | TTCTGCACATCACATTCATCGGGGGCCATCCATCGCGCCCTGAGA |
| | | | GTGGGTGAACCTGGTGCCCGAAACATGTGGGCCCCCGATGCTATC |
| | | | AGGACTGGCT |

(i) Preparation of Multiplex Pre-PC Composition Including Two Pre-PC Oligonucleotides for Each Target Nucleic Acid (Hereinafter Referred to as “Multiplex Pre-PC Composition (i)”):

The two pre-PC oligonucleotides are two pre-PC TOs for each of four target nucleic acids and have the sequences given in Table 7.

Multiplex pre-PC composition (i) including NG pre-PC TO-1 (SEQ ID NO: 19), NG pre-PC TO-2 (SEQ ID NO: 20), CT pre-PC TO-1 (SEQ ID NO: 31), CT pre-PC TO-2 (SEQ ID NO: 32), HBB pre-PC TO-1 (SEQ ID NO: 33), HBB pre-PC TO-2 (SEQ ID NO: 34), PSX pre-PC TO-1 (SEQ ID NO: 35), and PSX pre-PC TO-2 (SEQ ID NO: 36) each at a concentration of 6.0×10⁸ copies/μl.

(ii) Preparation of Multiplex Pre-PC Composition Including Three Pre-PC Oligonucleotides for Each Target Nucleic Acid (Hereinafter Referred to as “Multiplex Pre-PC Composition (ii)”):

The three pre-PC oligonucleotides are two pre-PC TOs and one pre-PC LO for each of four target nucleic acids and have the sequences given in Table 7.

Multiplex pre-PC composition (ii) including NG pre-PC TO-1 (SEQ ID NO: 21), NG pre-PC TO-2 (SEQ ID NO: 22), NG pre-PC LO-1 (SEQ ID NO: 23), CT pre-PC TO-1 (SEQ ID NO: 37), CT pre-PC TO-2 (SEQ ID NO: 38), CT pre-PC LO-1 (SEQ ID NO: 39), HBB pre-PC TO-1 (SEQ ID NO: 40), HBB pre-PC TO-2 (SEQ ID NO: 41), HBB pre-PC LO-1 (SEQ ID NO: 42), PSX pre-PC TO-1 (SEQ ID NO: 43), PSX pre-PC TO-2 (SEQ ID NO: 44), PSX pre-PC LO-1 (SEQ ID NO: 45) each at a concentration of 3.0×10⁸ copies/μl was prepared.

TABLE 7
Multiplex pre-PC Oligonucleotide

| pre-PC | Seq | | pre-PC | |
|---|---|---|---|---|
| composition | ID | | oligonucleotide | |
| TYPE | No. | Target | TYPE | Sequence (5′ to 3′) |
| Multiplex | 19 | NG | pre-PC | CGAAATTGCCGCCACTGCTTCCTACCGCTTCGGTAGT |
| pre-PC | | | TO-1 | CGAACGCAGTCAGAAACG |
| composition | 20 | NG | pre-PC | TCGTTTCAGCCAAGCGGCAGACATGATGGCGTGTTCG |
| (i) | | | TO-2 | CGTTTCTGACTGCGTTC |
| | 31 | CT | pre-PC | CTTTTTCCGCATCCAAACCAATTGTAATAGAAGCATT |
| | | | TO-1 | GGTTGCCAACTTTGATATTATCAGCAAAGAC |
| | 32 | CT | pre-PC | TTAAAGCTCAAAATAAAAAAGAGTTTATTTTTGGGAA |
| | | | TO-2 | ATTCACTGAAAACTGCGTCTTTGCTGATAATATCAAA |
| | | | | GT |
| | 33 | HBB | pre-PC | CCTTCAGTTAGCTTTGATCTTGTTTATTTCTTGTCTT |
| | | | TO-1 | CTGCTAGGAGGGCTTTAAGACTTTTTGATG |
| | 34 | HBB | pre-PC | CGGAATCTCTGGGACACAGCTTAGACAGTGTTAAGAG |
| | | | TO-2 | AGCCCACATCAAAAAGTCTTAAAGCCC |
| | 35 | PSX | pre-PC | AGCCAGTCCTGATAGCATCAGAAACCCACATGTTTCG |
| | | | TO-1 | GGCACCAGGTTCACCCACTC |
| | 36 | PSX | pre-PC | TTCTGCACATCACATTCATCCACACCCATCCATCGCG |
| | | | TO-2 | CCCTGAGAGTGGGTGAACCTGG |
| Multiplex | 21 | NG | pre-PC | CTGCGTTCGACTACCGAAGCGGTAGGAAGCAGTGGCG |
| pre-PC | | | TO-1 | GCAATTTCG |
| composition | 22 | NG | pre-PC | TCGTTTCAGCCAAGCGGCAGACATGATGGCGTGTTCG |
| (ii) | | | TO-2 | CGTTTCTGA |
| | 23 | NG | pre-PC | CGGTAGTCGAACGCAGTCAGAAACGCGAACACG |
| | | | LO-1 | |
| | 37 | CT | pre-PC | TGATAATATCAAAGTTGGCAACCAATGCTTCTATTAC |
| | | | TO-1 | AATTGGTTTGGATGCGGAAAAAG |
| | 38 | CT | pre-PC | TTAAAGCTCAAAATAAAAAAGAGTTTATTTTTGGGAA |
| | | | TO-2 | ATTCACTGAAAACTGCGTCTTTG |
| | 39 | CT | pre-PC | GTTGCCAACTTTGATATTATCAGCAAAGACGCAGTTT |
| | | | LO-1 | TCAG |
| | 40 | HBB | pre-PC | TCTTAAAGCCCTCCTAGCAGAAGACAAGAAATAAACA |
| | | | TO-1 | AGATCAAAGCTAACTGAAGG |
| | 41 | HBB | pre-PC | CGGAATCTCTGGGACACAGCTTAGACAGTGTTAAGAG |
| | | | TO-2 | AGCCCACATCAAAAA |
| | 42 | HBB | pre-PC | TGCTAGGAGGGCTTTAAGACTTTTTGATGTGGGCTCT |
| | | | LO-1 | CT |
| | 43 | PSX | pre-PC | AACCTGGTGCCCGAAACATGTGGGTTTCTGATGCTAT |
| | | | TO-1 | CAGGACTGGCT |
| | 44 | PSX | pre-PC | TTCTGCACATCACATTCATCCACACCCATCCATCGCG |
| | | | TO-2 | CCCTGAGAGTGGG |
| | 45 | PSX | pre-PC | TCGGGCACCAGGTTCACCCACTCTCAGGGCG |
| | | | LO-1 | |

(1-3) Preparation of Comparative Positive Control Composition

A plasmid DNA positive control which is widely used in the art was prepared as s comparative positive control.

(a) Preparation of Comparative Positive Control Composition for NG Target Nucleic Acid

A sequence including the NG gene target nucleic acid sequence of SEQ ID NO: 1 was inserted into vector pIDTSMART-AMP (GenBank: MP690161.1) to construct a plasmid DNA positive control for the NG target nucleic acid.

A comparative positive control composition including the plasmid DNA positive control at a concentration of 2.0×10⁵ copies/μl was serially 10-fold diluted with a TE to prepare plasmid DNA positive control compositions having five concentrations in total (concentration 1: 2.0×10⁵ copies/μl, concentration 2: 2.0×10⁴ copies/μl, concentration 3: 2.0×10³ copies/μl, concentration 4: 2.0×10² copies/μl, and concentration 5: 2.0×10¹ copies/μl).

(b) Preparation of Multiplex Comparative Positive Control Composition for Multiplex Target Nucleic Acid

Sequences including the target nucleic acid sequences of SEQ ID NOS: 1-4 (NG, CT, HBB, and PSX) were inserted to four vector pIDTSMART-AMP (GenBank: MP690161.1) to prepare plasmid DNA positive controls for the four target nucleic acids, respectively.

A multiplex comparative positive control composition comprising the prepared plasmid DNA positive control for CT target nucleic acid at a concentration of 2.0×10⁶ copies/μl and the prepared plasmid DNA controls for NG, HBB, and PSX target nucleic acids each at a concentration of 2.0×10⁵ copies/μl was prepared.

### Example 2: Positive Control Reaction

Examination was made of the availability of the pre-PC compositions according to the present disclosure in implementing positive control reactions. In this regard, positive control reactions were conducted for the NG target nucleic acid, using the NG target nucleic acid-detecting compositions and NG pre-PC compositions (i) to (iv) with different concentrations, prepared in Example 1. The results were compared with those of the positive control reactions using the comparative positive control for the NG target nucleic acid.

(2-1) Positive Control Reaction Using NG Pre-PC Composition

First, 18 tubes were prepared. To the 18 tubes, 5 μl of each of NG pre-PC compositions (i) to (iv) having the various concentrations, prepared in Example (1-2), were added, respectively, followed by 4 pmoles of an NG amplification forward primer (SEQ ID NO: 5), 4 pmoles of an NG amplification reverse primer (SEQ ID NO: 6), 4 pmoles of an NG detection probe (SEQ ID NO: 7), and 5 μl of EM1 (Cat No. SD10245Z, Seegene, Inc.) to afford a reaction mixture having a final volume of 20 μl per tube. The reaction mixtures were subjected to real-time PCR.

The tubes including the reaction mixture were positioned in a real-time thermal cycler (CFX96, Bio-Rad). The real-time PCR started with denaturation of the reaction mixture at 95° C. for 15 min, followed by 45 cycles of 95° C. for 10 sec, 60° C. for 60 sec, and 72° C. for sec. Signal detection was made at 60° C. every cycle. Ct values were calculated using an auto-calculated single threshold. All the experiments were conducted in duplicate and average Ct values were obtained from the two measurements, along with a difference in Ct value between reactions (ΔCt). A negative control reaction was carried out with a reaction mixture including 5 μl of distilled water instead of the pre-PC composition in the same condition.

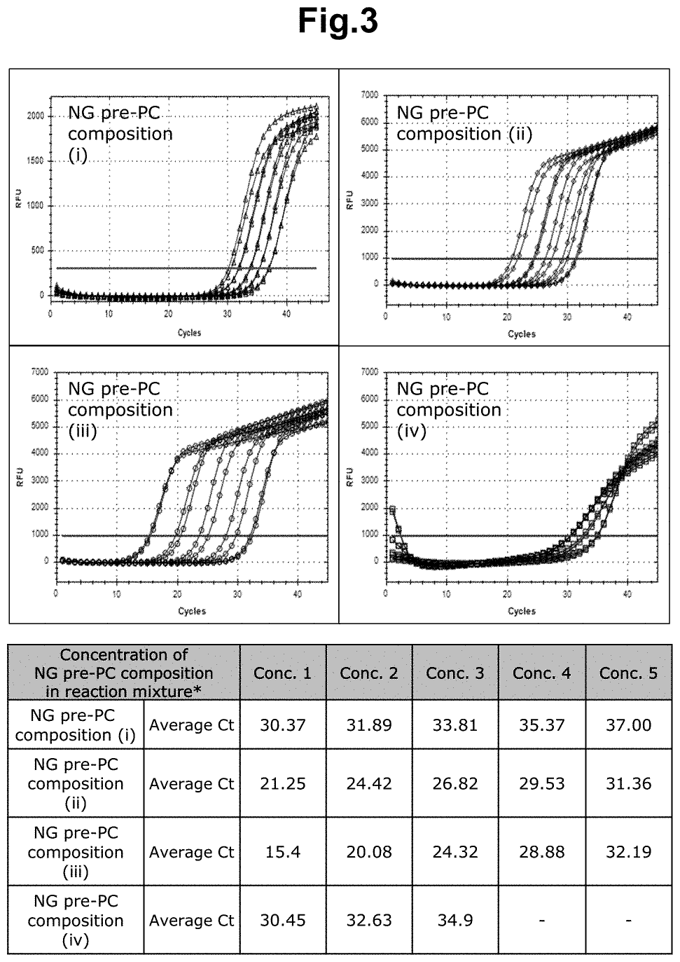

As shown in FIG. 3, Ct values ranging from 15 to 37 were obtained when the 18-type NG pre-PC compositions were used to conduct positive control reactions for an NG target nucleic acid. In contrast, no signals were detected from the negative control reaction.

The data thus obtained indicate that although no complete-positive control sequences are given in an initial reaction phase, the pre-PC composition for allowing the production of a complete-positive control sequence in a reaction procedure can be used as a positive control for a target nucleic acid.

(2-2) Positive Control Reaction Using Comparative Positive Control

Reaction mixtures were prepared and reacted in the same manner as in Example (2-1), with the exception of using the comparative positive control compositions having five concentrations, prepared in Example (1-3), for the NG target nucleic acid, instead of the pre-PC compositions.

The reaction mixtures, each having a final volume of 20 μl, included the plasmid DNA positive control in amounts of 10⁶ copies, 10³ copies, 10⁴ copies, 10³ copies, and 10² copies, respectively. A negative control reaction was carried out with a reaction mixture including 5 μl of distilled water instead of the plasmid DNA positive control compositions in the same condition.

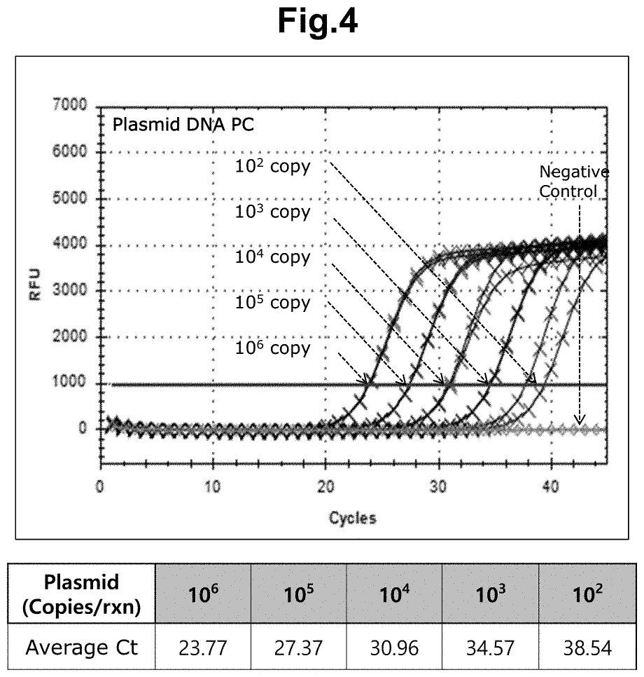

As shown in FIG. 4, Ct values ranging from 23 to 39 were obtained when the plasmid DNA positive control composition having the five concentrations were used to conduct positive control reactions for the target nucleic acid. In contrast, no signals were detected from the negative control reaction.

From the data, it was understood that the target nucleic acid-detecting compositions used in this Example can work normally in the presence of a target nucleic acid sequence to provide signals indicative of the existence of the target nucleic acid sequence.

### Example 3: Ct Value Change Rate According to Pre-Positive Control Composition

Ct value change rates according to pre-PC oligonucleotide concentrations of the pre-PC compositions were calculated using the results of Example 2, and then compared to those according to plasmid DNA concentrations in the comparative positive control composition.

(3-1) Ct Value Change Rate According to Concentration of Pre-PC Composition

Ct value change rates according to the number of copies were calculated on the basis of results of the positive control reactions using the NG pre-PC compositions having various concentrations (three or five concentrations), prepared in (2-1) of Example 2.

In order to perform a linear regression on the linear functions, the Ct change rates (y axis) were plotted against log-scale numbers of each copy (x axis).

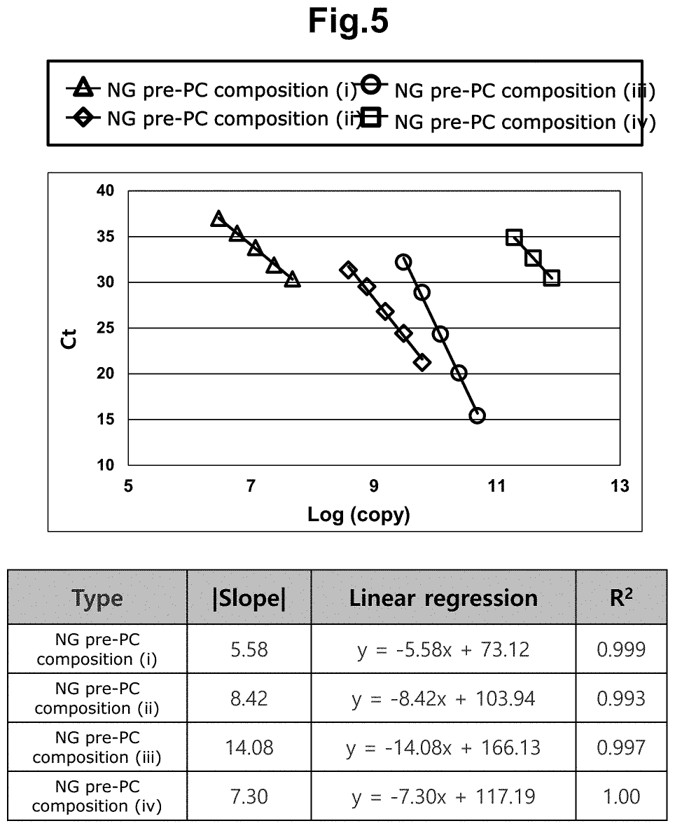

As shown in FIG. 5, the following functions were obtained for NG pre-PC compositions (i) to (iv).

NG pre-PC composition(i):y=−5.58x+73.12(R²=0.999)

NG pre-PC composition(ii):y=−8.42x+103.94(R²=0.993)

NG pre-PC composition(iii):y=−14.08x+166.13(R²=0.997)

NG pre-PC composition(iv):y=−7.30x+117.19(R²=1.00)

(3-2) Ct Value Change Rate According to Comparative Positive Control Concentration

Ct value change rates according to the number of copies were calculated on the basis of results of the positive control reactions using the comparative positive control composition having five concentrations, prepared in (2-2) of Example 2.

Linear regression analysis was performed in the same manner as in Example (3-1).

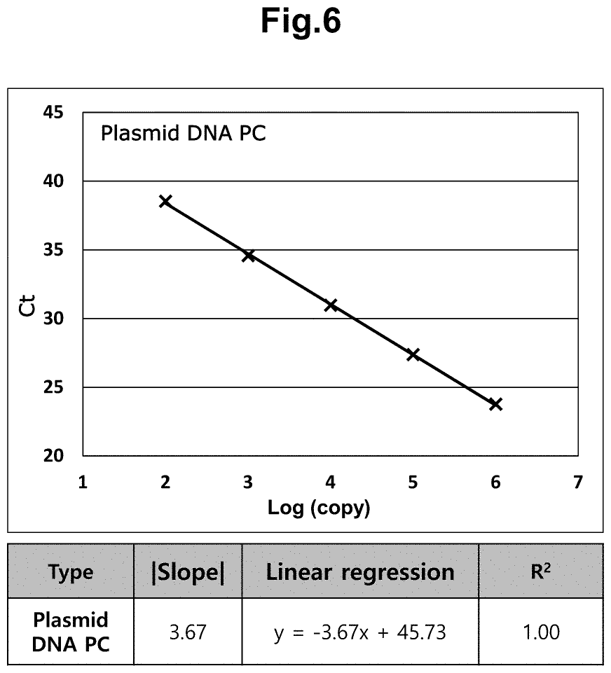

As shown in FIG. 6, the following function was obtained.

y=−3.67x+45.73(R²=1.00)

(3-3) Comparison of Ct Value Change Rate Between Pre-PC Composition and Comparative Positive Control

Comparison of the two functions indicated that the pre-PC compositions exhibited larger Ct value changes with concentrations. For example, when the concentration was 10-fold diluted, the pre-PC compositions exhibited a Ct value difference of about 5.58-14.08 while the plasmid DNA exhibited a Ct value difference of about 3.67.

With the assumption that a positive control is detected to have a Ct value of 25 in a correctly performed experiment, when 20 μl of a different PCR reaction is contaminated with 1/100 of the concentration of the pre-PC composition (iii) or the plasmid DNA positive control (i.e., 0.05 μl of the pre-PC composition (iii) or the plasmid DNA positive control is included), the Ct value attributed to the pre-PC composition (iii) or the plasmid DNA positive control will be 53.16 or 36.16, respectively. In a PCR reaction conducted with 45 to 50 cycles, the results are not detected to be positive upon contamination of the pre-PC composition (iii) whereas the plasmid DNA positive control may incur a false positive result from the Ct value of 36.16. In experiments relevant thereto that the present inventors conducted, no signals were detected when the pre-PC composition was contaminated at a rate of 1/100 of the concentration thereof whereas signals appeared upon contamination of the plasmid DNA positive control (not shown).

These data imply that the pre-PC composition according to the present disclosure is more advantageous in controlling the contamination that might be generated upon preparation of a positive control or during experiments than conventional plasmid DNA positive controls.

In addition, in the case where pre-PC oligonucleotides are separately provided for pre-PC compositions, a complete-positive control can be produced only when all of the pre-PC oligonucleotides are contaminated simultaneously. Therefore, the pre-PC composition may be less prone to contamination than the plasmid DNA positive control.

### Example 4: Assay for Operability of Target Nucleic Acid-Detecting Oligonucleotide

Some of the various pre-PC compositions prepared in this Example can produce intermediate-positive control oligonucleotides including complementary sequences to probes. Moreover, since pre-PC TO can act like a primer, the probe oligonucleotides hybridized with the intermediate-positive control oligonucleotides may be cleaved with Taq polymerase to generate a signal without involving the target nucleic acid-detecting forward and reverse primer oligonucleotides therein.

Even in the case where the primers for target nucleic acid amplification do not work properly, if the pre-PC TO operates like a primer to allow the cleavage of probe oligonucleotides by Taq polymerase and the generation of a fluorescent signal as high as or higher than a threshold irrespective of the production and/or amplification of a complete-positive control oligonucleotide, the pre-PC composition cannot be used as a positive control.

The pre-PC composition according to the present disclosure may comprise a pre-PC oligonucleotide at a concentration that is sufficient to allow the cleavage of a probe oligonucleotide by Taq polymerase, but is not sufficient to generate a fluorescent signal as high as or higher than a threshold.

A pair of primers for target nucleic acid amplification should both operate in the pre-PC composition, in order that a complete-positive control produced from the pre-PC composition is sufficiently amplified and the cleavage of a probe oligonucleotide leads to the generation of a fluorescent signal at a threshold level or higher. If any of the primers does not work normally, the complete-positive control is not effectively amplified, with the consequent failure of fluorescent signal generation at a threshold level or higher.

In order to examine whether the pre-PC composition of the present disclosure can be used for evaluating the operability of the target nucleic acid-detecting oligonucleotides included in the target nucleic acid-detecting composition, positive control reactions were performed using NG pre-PC composition (ii) of concentration 1 (6.0×10⁸ copies/μl) and NG pre-PC composition (iii) of concentration 4 (3.0×10⁸ copies/μl), prepared the above Example in a condition under which some NG target nucleic acid-detecting oligonucleotides cannot operate.

As conditions under which the NG target nucleic acid-detecting oligonucleotides do not operate, target nucleic acid-detecting compositions, which comprise only either of the paired primers ((ii) and (iii) in Table 8) and a target nucleic acid-detecting composition which includes neither of the paired primers for target nucleic acid amplification ((iv) in Table 8) were prepared as shown in Table 8. As a comparative control, a target nucleic acid-detecting composition including all of the paired primers was prepared for a condition under which all the target nucleic acid-detecting oligonucleotides can operate ((i) in Table 8).

In addition, a positive control reaction is performed using the plasmid DNA positive control of concentration 1 (2.0×10⁵ copies/μl), prepared in Example (1-3), instead of the pre-PC composition in the same condition. A negative control reaction was performed using a reaction mixture including 5 μl of distilled water instead of the pre-PC composition.

TABLE 8
Target Nucleic Acid-Detecting Composition of Example 4

| | Target nucleic acid-detecting |
|---|---|
| No. | oligonucleotide composition |
| i | Forward primer (SEQ ID NO: 5) |
| | Reverse primer (SEQ ID NO: 6) |
| | Probe (SEQ ID NO: 7) |
| ii | Forward primer (SEQ ID NO: 5) |
| | Probe (SEQ ID NO: 7) |
| iii | Reverse primer (SEQ ID NO: 6) |
| | Probe (SEQ ID NO: 7) |
| iv | Probe (SEQ ID NO: 7) |

Reaction mixtures were prepared using the four-type target nucleic acid-detecting compositions in Table 8, NG pre-PC composition (ii) of concentration 1 (6.0×10⁸ copies/μl), NG pre-PC composition (iii) of concentration 1 (2.4×10⁹ copies/μl), and a plasmid DNA positive control of concentration 4 (3.0×10⁸ copies/μl) and positive control reactions were performed thereon in the same manner as in the “positive control reaction of Example 2”.

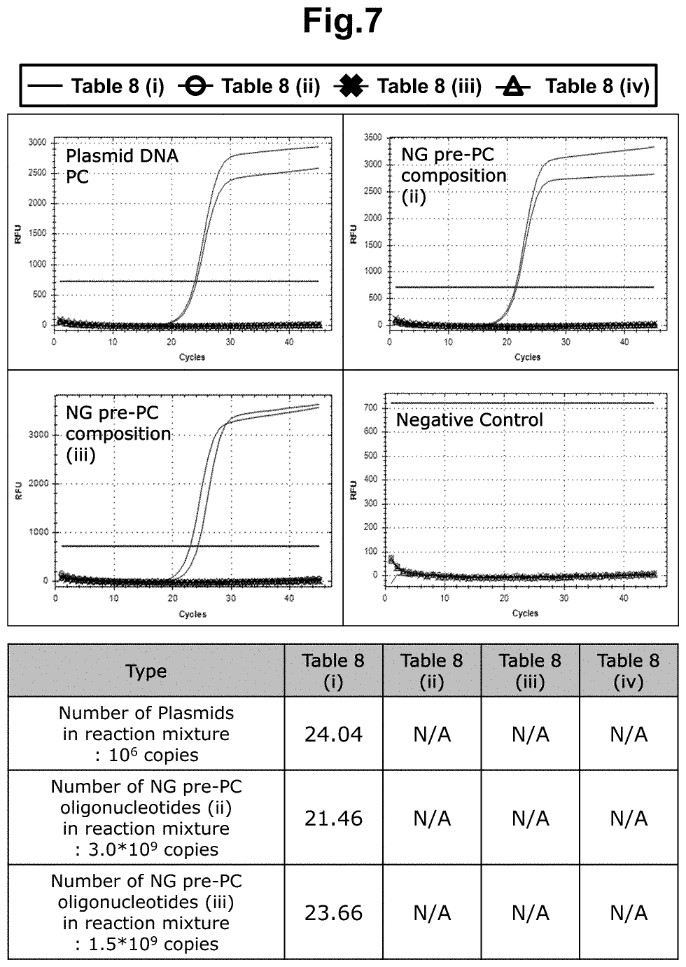

As shown in FIG. 7, signals were detected only when all of the primers for target nucleic acid amplification and the probe operate ((i) in Table 8), but not detected when at least one of the primers for target nucleic acid amplification does not operate ((ii) to (iv) in Table 8). No signals were detected in the negative control reaction.

From the assay for operability of pre-PC compositions, it was understood that Ct values similar to the reaction results of Table 8 (i) in which all the primers for target nucleic acid amplification operated could not be obtained with only the pre-PC compositions which had much smaller numbers of oligonucleotides (3.0×10⁹ copies to 1.5×10⁹ copies in reaction mixture) than that of the target nucleic acid-detecting oligonucleotides (2.4×10¹² copies in reaction mixture). That is, neither could the complete-positive control be sufficiently amplified, nor could a fluorescent signal be generated at a threshold level or higher. This means that only when the pre-PC composition according to the present disclosure operates together with the primers for nucleic acid amplification, Ct values can be obtained.

Therefore, the data obtained above indicate that the pre-PC composition of the present disclosure can be used for assaying the operability of target nucleic acid-detecting oligonucleotides included in a target nucleic acid-detecting composition.

### Example 5: Multiplex Positive Control Reaction Using Multiplex Pre-PC Composition

The multiplex pre-PC composition according to the present disclosure was assayed for availability in implementing a multiplex positive control reaction. To this end, the multiplex target nucleic acid-detecting composition prepared in Example (1-1) and the multiplex pre-PC compositions (i) to (ii) prepared in Example (1-2) were used to conduct multiplex positive control reactions on four target nucleic acids. In addition, comparison was made with the multiplex positive control reaction using the multiplex comparative positive control composition prepared in Example (1-3).

(5-1) Multiplex Positive Control Reaction Using Multiplex Pre-PC Composition

Reaction mixtures were prepared using the multiplex target nucleic acid-detecting composition prepared in Example (1-1) and the multiplex pre-PC compositions (i) to (ii) for four target nucleic acids prepared in Example (1-2) and positive control reactions were performed thereon in the same manner as in the “positive control reaction of Example 2”.

In 20 μl of the final reaction mixture, pre-PC oligonucleotides of multiplex pre-PC composition (i) and multiplex pre-PC composition (ii) were included in an amount of 3.0×10⁹ copies and 1.5×10⁹ copies, respectively. For a negative control reaction, a reaction mixture including 5 μl of distilled water instead of the pre-PC composition was used.

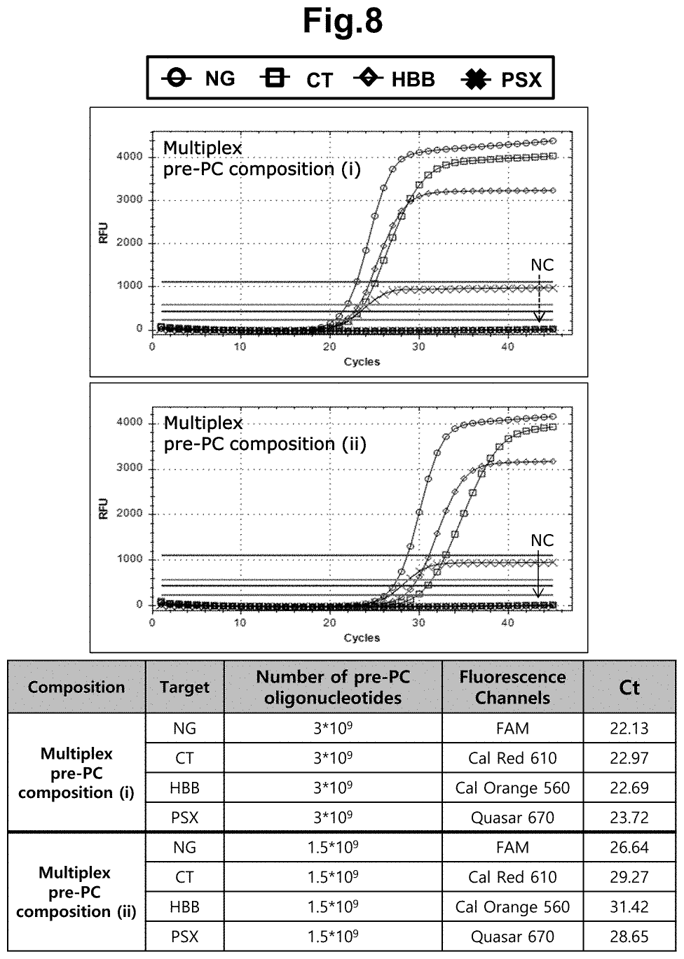

As shown in FIG. 8, when a multiplex positive control reaction was performed on the four target nucleic acids using multiplex pre-PC composition (i), Ct values ranging from 22.1 to 23.7 were obtained for the four target nucleic acids. In a multiplex positive control reaction performed on the four target nucleic acids using multiplex pre-PC composition (ii), Ct values of 26.6 to 31.4 were obtained for the four target nucleic acids. In contrast, no signals were detected in the negative control reaction.

From the results, it is understood that the pre-PC composition according to the present disclosure can be used for performing a positive control reaction on a plurality of target nucleic acids.

(5-2) Multiplex Positive Control Reaction Using Multiplex Comparative Positive Control

Reaction mixtures were prepared using the multiplex target nucleic acid-detecting composition prepared in Example (1-1) and the comparative positive control composition for multiplex target nucleic acids prepared in Example (1-3), and positive control reactions were performed thereon in the same manner as in the “positive control reaction of Example 2”.

In 20 μl of the final reaction mixture, the plasmid DNA positive control was included in an amount of 10⁷ copies for the CT target nucleic acid and in an amount of 10⁶ copies for each of NG, HBB, and PSX target nucleic acids. For a negative control reaction, a reaction mixture including 5 μl of distilled water instead of the multiplex comparative control composition was used.

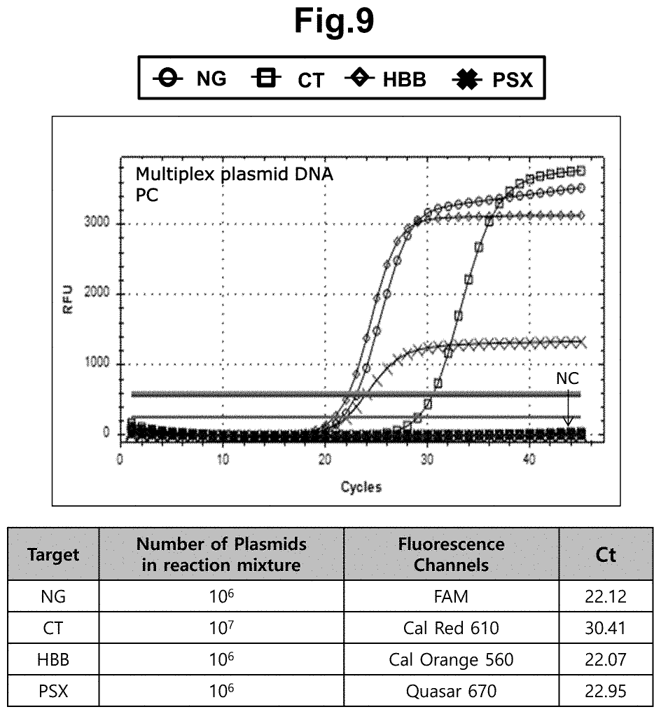

As shown in FIG. 9, when a multiplex positive control reaction was performed on the four target nucleic acids using the multiplex comparative positive control composition, Ct values ranging from 22.1 to 30.4 were obtained for the four target nucleic acids. In contrast, no signals were detected in the negative control reaction.

The data obtained above imply that the multiplex target nucleic acid-detecting composition used in this Example can operate normally in the presence of the multiplex comparative positive control sequences, thereby generating signals indicative of the existence of target nucleic acid sequences.

1.-32. (canceled)

33. A method for conducting a positive control reaction for a target nucleic acid-detecting composition by using a pre-positive control composition, the method comprising the steps of:
(a) mixing the target nucleic acid-detecting composition and the pre-positive control composition, wherein,
the target nucleic acid-detecting composition comprises target nucleic acid-detecting oligonucleotides comprising a forward primer oligonucleotide and a reverse primer oligonucleotide for target nucleic acid amplification;
the pre-positive control composition comprises a pre-positive control oligonucleotide, the pre-positive control oligonucleotide comprising a partial sequence of a complete-positive control oligonucleotide for the target nucleic acid sequence;
the pre-positive control oligonucleotide comprises a sequence overlapping with at least one oligonucleotide selected from among the target nucleic acid-detecting oligonucleotides and a different pre-positive control oligonucleotide, wherein the overlapping sequence of the pre-positive control oligonucleotide is identical or complementary to an overlapping sequence of the at least one oligonucleotide;
the pre-positive control composition comprises one or more pre-positive control oligonucleotides; and
the one or more pre-positive control oligonucleotides are selected so as to produce the complete-positive control by assembling (i) the pre-positive control oligonucleotides or (ii) the one or more pre-positive control oligonucleotides and the at least one of the target nucleic acid-detecting oligonucleotides;
(b) producing the complete-positive control oligonucleotide through an assembly process between different oligonucleotides, the assembly process comprising the steps of:
(b-1) hybridizing at least one complementary combination among combinations of two oligonucleotides comprising an overlapping sequence identical or complementary to each other in the mixture;
(b-2) extending either or both of the two oligonucleotides hybridized with each other; and
(b-3) producing the complete-positive control oligonucleotide;
wherein a product resulting from the extending step is the complete-positive control oligonucleotide or an intermediate-positive control oligonucleotide, and when the intermediate-positive control oligonucleotide is produced, at least one round of a step of denaturing double-stranded nucleic acids and steps (b-1) and (b-2) is further repeated to produce the complete-positive control oligonucleotide; and
(c) amplifying the complete-positive control oligonucleotide using the forward primer oligonucleotide and the reverse primer oligonucleotide.

34. The method of claim 33, wherein the one or more pre-positive control oligonucleotides comprise a sequence overlapping with at least one of the target nucleic acid-detecting oligonucleotides.

35. The method of claim 33, wherein the complete-positive control oligonucleotide is produced in a double-stranded form, any one strand of which comprises a sequence of the one or more pre-positive control oligonucleotides.

36. The method of claim 33, wherein the 3′-end of the pre-positive control oligonucleotide is extendable therefrom or blocked to prevent extension therefrom.

37. The method of claim 33, wherein the combinations have overlapping sequences being neither identical nor complementary to each other.

38. The method of claim 33, wherein the pre-positive control oligonucleotide is a pre-positive control templating oligonucleotide or a pre-positive control linking oligonucleotide;
the pre-positive control templating oligonucleotide comprising a sequence that overlaps with at least one of the forward primer oligonucleotide or reverse primer oligonucleotide and not comprising sequences that overlap totally with the full sequences of the forward primer oligonucleotide and the reverse primer oligonucleotide, and
the pre-positive control linking oligonucleotide comprising sequences overlapping with two different pre-positive control oligonucleotides at its 3′-end and 5′-end and not comprising a sequence overlapping with the forward primer oligonucleotide or the reverse primer oligonucleotide.

39. The method of claim 33, wherein the pre-positive control composition comprises one to five pre-positive control oligonucleotides.

40. The method of claim 38, wherein the pre-positive control composition comprises one pre-positive control oligonucleotide, wherein the one pre-positive control oligonucleotide comprises one pre-positive control templating oligonucleotide.

41. The method of claim 40, wherein the one pre-positive control templating oligonucleotide comprises sequences overlapping with the forward primer oligonucleotide and reverse primer oligonucleotide at its 3′-end and 5′-end and does not comprise sequences overlapping totally with the full sequences of the forward primer oligonucleotide and the reverse primer oligonucleotide.

42. The method of claim 38, wherein the pre-positive control composition comprises (i) two pre-positive control oligonucleotides, wherein the two pre-positive control oligonucleotides consist of two pre-positive control templating oligonucleotides, (ii) three pre-positive control oligonucleotides, wherein the three pre-positive control oligonucleotides consist of two pre-positive control templating oligonucleotides and one pre-positive control linking oligonucleotide, or (iii) four pre-positive control oligonucleotides, wherein the four pre-positive control oligonucleotides consist of two pre-positive control templating oligonucleotides and two pre-positive control linking oligonucleotides.

43. The method of claim 42, wherein the two pre-positive control templating oligonucleotides consist of a first pre-positive control templating oligonucleotide comprising a sequence overlapping with the forward primer oligonucleotide and a second pre-positive control templating oligonucleotide comprising a sequence overlapping with the reverse primer oligonucleotide.

44. A pre-positive control composition, comprising a pre-positive control oligonucleotide;
wherein the pre-positive control oligonucleotide comprises a sequence overlapping with at least one oligonucleotide selected from among target nucleic acid-detecting oligonucleotides included in a target nucleic acid-detecting composition and a different pre-positive control oligonucleotide, wherein the overlapping sequence of the pre-positive control oligonucleotide is identical or complementary to an overlapping sequence of the at least one oligonucleotide;
wherein the pre-positive control composition comprises one or more pre-positive control oligonucleotides;
wherein the one or more pre-positive control oligonucleotides are selected so as to produce the complete-positive control oligonucleotide by assembling (i) the pre-positive control oligonucleotides or (ii) the one or more pre-positive control oligonucleotides and the at least one of the target nucleic acid-detecting oligonucleotides;
wherein a mixture of the pre-positive composition and the target nucleic acid-detecting composition comprises combinations of two oligonucleotides comprising an overlapping sequence identical or complementary to each other; and
whereby the pre-positive control composition is capable of producing a complete-positive control oligonucleotide for a positive control reaction for the target nucleic acid-detecting composition.

45. The composition of claim 44, wherein the one or more pre-positive control oligonucleotides comprise a sequence overlapping with at least one of the target nucleic acid-detecting oligonucleotides.

46. The composition of claim 44, wherein the complete-positive control oligonucleotide is in a double-stranded form, any one strand of which comprises a sequence of the one or more pre-positive control oligonucleotides.

47. The composition of claim 44, wherein the 3′-end of the pre-positive control oligonucleotide is extendable therefrom or blocked to prevent extension therefrom.

48. The composition of claim 44, wherein the combinations have overlapping sequences being neither identical nor complementary to each other.

49. The composition of claim 44, wherein the target nucleic acid-detecting oligonucleotides comprise a forward primer oligonucleotide and a reverse primer oligonucleotide for target nucleic acid amplification.

50. The composition of claim 49, wherein the pre-positive control oligonucleotide is a pre-positive control templating oligonucleotide or a pre-positive control linking oligonucleotide;
the pre-positive control templating oligonucleotide comprising a sequence that overlaps with at least one of the forward primer oligonucleotide or reverse primer oligonucleotide and not comprising sequences that overlap totally with the full sequences of the forward primer oligonucleotide and the reverse primer oligonucleotide, and
the pre-positive control linking oligonucleotide comprising sequences overlapping with two different pre-positive control oligonucleotides at its 3′-end and 5′-end and not comprising a sequence overlapping with the forward primer oligonucleotide or the reverse primer oligonucleotide.

51. The composition of claim 44, wherein the pre-positive control composition comprises one to five pre-positive control oligonucleotides.

52. The composition of claim 50, wherein the pre-positive control composition comprises one pre-positive control oligonucleotide, wherein the one pre-positive control oligonucleotide comprises one pre-positive control templating oligonucleotide.

53. The composition of claim 52, wherein the one pre-positive control templating oligonucleotide comprises sequences overlapping with the forward primer oligonucleotide and reverse primer oligonucleotide at its 3′-end and 5′-end and does not comprise sequences overlapping totally with the full sequences of the forward primer oligonucleotide and the reverse primer oligonucleotide.

54. The composition of claim 50, wherein the pre-positive control composition comprises (i) two pre-positive control oligonucleotides, wherein the two pre-positive control oligonucleotides consist of two pre-positive control templating oligonucleotides, (ii) three pre-positive control oligonucleotides, wherein the three pre-positive control oligonucleotides consist of two pre-positive control templating oligonucleotides and one pre-positive control linking oligonucleotide, or (iii) four pre-positive control oligonucleotides, wherein the four pre-positive control oligonucleotides consist of two pre-positive control templating oligonucleotides and two pre-positive control linking oligonucleotides.

55. The composition of claim 52, wherein the two pre-positive control templating oligonucleotides consist of a first pre-positive control templating oligonucleotide comprising a sequence overlapping with the forward primer oligonucleotide and a second pre-positive control templating oligonucleotide comprising a sequence overlapping with the reverse primer oligonucleotide.

56. A kit for conducting a positive control reaction for a target nucleic acid-detecting composition, the kit comprising:
(a) a target nucleic acid-detecting composition, wherein the target nucleic acid-detecting composition comprises target nucleic acid-detecting oligonucleotides comprising a forward primer oligonucleotide and a reverse primer oligonucleotide for target nucleic acid amplification; and
(b) a pre-positive control composition comprising a pre-positive control oligonucleotide,
wherein the pre-positive control oligonucleotide comprises a sequence overlapping with at least one oligonucleotide selected from among target nucleic acid-detecting oligonucleotides included in a target nucleic acid-detecting composition and a different pre-positive control oligonucleotide, wherein the overlapping sequence of the pre-positive control oligonucleotide is identical or complementary to an overlapping sequence of the at least one oligonucleotide;
wherein the pre-positive control composition comprises one or more pre-positive control oligonucleotides;
wherein the one or more pre-positive control oligonucleotides are selected so as to produce the complete-positive control oligonucleotide by assembling (i) the pre-positive control oligonucleotides or (ii) the one or more pre-positive control oligonucleotides and the at least one of the target nucleic acid-detecting oligonucleotides;
wherein a mixture of the pre-positive composition and the target nucleic acid-detecting composition comprises combinations of two oligonucleotides comprising an overlapping sequence identical or complementary to each other; and
whereby the pre-positive control composition is capable of producing a complete-positive control oligonucleotide for a positive control reaction for the target nucleic acid-detecting composition.


SEQUENCE LISTING (45 sequences)

SEQ ID NO: 1 - DNA, 190 bp, Artificial Sequence
  target NG
cgaaattgccgccactgcttcctaccgcttcggtaatacagtcccgcgcatcagctatgcccatggtttcgactttgtcgaacgcagtcagaaacgcgaacataccagctatgatcaaatcatcgccggtgtcgattacgatttttccaagcgcacttccgccatcatgtctgccgcttggctgaaacga

SEQ ID NO: 2 - DNA, 256 bp, Artificial Sequence
  target CT
ctttttccgcatccaaaccaattgtaatagaagcattggttgatgaattattggagactgttaaagatattccatcagaagctgtcattttggctgcgacaggtgttgatgttgtcccaaggattatttgctggtccttgagcggctctgtcatttgcccaactttgatattatcagcaaagacgcagttttcagtgttatacaaataaaaaccagaatttcccattttaaaactcttttttattttgagctttaa

SEQ ID NO: 3 - DNA, 191 bp, Artificial Sequence
  target HBB
ccttcagttagctttgatcttgtttatttcttgtcttctgctagatttagggttggtttgctcttggttctctggttcttttagttgtgacattaggttgttaatttgagggctttaagactttttgatgtgggcatttagtgtataaatttctctcttaacactgtctaagctgtgtcccagagattccg

SEQ ID NO: 4 - DNA, 192 bp, Artificial Sequence
  target PSX
agccagtcctgatagcatcagaaacccacatgttctgaataggctggctcaactgcggtacagacgcaccaggttcacccactctcagctgcatgacctggagcgccttttccaagagactcgctaccccagcttgcgagcaaggagggatcttgcacgatggatgggtgtggatgaatgtgatgtgcagaa

SEQ ID NO: 5 - DNA, 31 bp, Artificial Sequence
  NG F-primer
  misc_feature (18)..(22): n denotes deoxyinosine
cgaaattgccgccactgnnnnntaccgcttc

SEQ ID NO: 6 - DNA, 31 bp, Artificial Sequence
  NG R-primer
  misc_feature (18)..(22): n denotes deoxyinosine
tcgtttcagccaagcggnnnnnatgatggcg

SEQ ID NO: 7 - DNA, 22 bp, Artificial Sequence
  NG probe
cgcgtttctgactgcgttcgac

SEQ ID NO: 8 - DNA, 38 bp, Artificial Sequence
  CT F-primer
  misc_feature (24)..(28): n denotes deoxyinosine
ctttttccgcatccaaaccaattnnnnnagaagcattg

SEQ ID NO: 9 - DNA, 41 bp, Artificial Sequence
  CT R-primer
  misc_feature (27)..(31): n denotes deoxyinosine
ttaaagctcaaaataaaaaagagtttnnnnntgggaaattc

SEQ ID NO: 10 - DNA, 34 bp, Artificial Sequence
  CT probe
ccaactttgatattatcagcaaagacgcagtttt

SEQ ID NO: 11 - DNA, 37 bp, Artificial Sequence
  HBB F-primer
  misc_feature (25)..(29): n denotes deoxyinosine
ccttcagttagctttgatcttgttnnnnncttgtctt

SEQ ID NO: 12 - DNA, 33 bp, Artificial Sequence
  HBB R-primer
  misc_feature (21)..(25): n denotes deoxyinosine
cggaatctctgggacacagcnnnnncagtgtta

SEQ ID NO: 13 - DNA, 28 bp, Artificial Sequence
  HBB probe
gagggctttaagactttttgatgtgggc

SEQ ID NO: 14 - DNA, 34 bp, Artificial Sequence
  PSX F-primer
  misc_feature (20)..(24): n denotes deoxyinosine
agccagtcctgatagcatcnnnnncccacatgtt

SEQ ID NO: 15 - DNA, 35 bp, Artificial Sequence
  PSX R-primer
  misc_feature (21)..(25): n denotes deoxyinosine
ttctgcacatcacattcatcnnnnnccatccatcg

SEQ ID NO: 16 - DNA, 31 bp, Artificial Sequence
  PSX probe
tcgggcaccaggttcacccactctcagggcg

SEQ ID NO: 17 - DNA, 92 bp, Artificial Sequence
  NG Complete-positive control oligonucleotide
tcgtttcagccaagcgggggggatgatggcgtgttcgcgtttctgactgcgttcgactaccgaagcggtaccccccagtggcggcaatttcg

SEQ ID NO: 18 - DNA, 58 bp, Artificial Sequence
  NG pre-PC composition (i), pre-PC TO
gggggatgatggcgtgttcgcgtttctgactgcgttcgactaccgaagcggtaccccc

SEQ ID NO: 19 - DNA, 55 bp, Artificial Sequence
  NG pre-PC composition (ii), pre-PC TO-1
cgaaattgccgccactgcttcctaccgcttcggtagtcgaacgcagtcagaaacg

SEQ ID NO: 20 - DNA, 54 bp, Artificial Sequence
  NG pre-PC composition (ii), pre-PC TO-2
tcgtttcagccaagcggcagacatgatggcgtgttcgcgtttctgactgcgttc

SEQ ID NO: 21 - DNA, 46 bp, Artificial Sequence
  NG pre-PC composition (iii), pre-PC TO-1
ctgcgttcgactaccgaagcggtaggaagcagtggcggcaatttcg

SEQ ID NO: 22 - DNA, 46 bp, Artificial Sequence
  NG pre-PC composition (iii), pre-PC TO-2
tcgtttcagccaagcggcagacatgatggcgtgttcgcgtttctga

SEQ ID NO: 23 - DNA, 33 bp, Artificial Sequence
  NG pre-PC composition (iii), pre-PC LO-1
cggtagtcgaacgcagtcagaaacgcgaacacg

SEQ ID NO: 24 - DNA, 30 bp, Artificial Sequence
  NG pre-PC composition (iv), pre-PC TO-1
aagcggtaggaagcagtggcggcaatttcg

SEQ ID NO: 25 - DNA, 32 bp, Artificial Sequence
  NG pre-PC composition (iv), pre-PC TO-2
acgccatcatgtctgccgcttggctgaaacga

SEQ ID NO: 26 - DNA, 42 bp, Artificial Sequence
  NG pre-PC composition (iv), pre-PC LO-1
actgcttcctaccgcttcggtagtcgaacgcagtcagaaacg

SEQ ID NO: 27 - DNA, 39 bp, Artificial Sequence
  NG pre-PC composition (iv), pre-PC LO-2
ggcagacatgatggcgtgttcgcgtttctgactgcgttc

SEQ ID NO: 28 - DNA, 121 bp, Artificial Sequence
  CT complete-positive control oligonucleotide
ctttttccgcatccaaaccaattgggggagaagcattggttgccaactttgatattatcagcaaagacgcagttttcagtgaatttcccacccccaaactcttttttattttgagctttaa

SEQ ID NO: 29 - DNA, 110 bp, Artificial Sequence
  HBB complete-positive control oligonucleotide
cggaatctctgggacacagcgggggcagtgttaagagagcccacatcaaaaagtcttaaagccctcctagcagaagacaagcccccaacaagatcaaagctaactgaagg

SEQ ID NO: 30 - DNA, 100 bp, Artificial Sequence
  PSX complete-positive control oligonucleotide
ttctgcacatcacattcatcgggggccatccatcgcgccctgagagtgggtgaacctggtgcccgaaacatgtgggcccccgatgctatcaggactggct

SEQ ID NO: 31 - DNA, 68 bp, Artificial Sequence
  CT pre-PC composition (i), pre-PC TO-1
ctttttccgcatccaaaccaattgtaatagaagcattggttgccaactttgatattatcagcaaagac

SEQ ID NO: 32 - DNA, 76 bp, Artificial Sequence
  CT pre-PC composition (i), pre-PC TO-2
ttaaagctcaaaataaaaaagagtttatttttgggaaattcactgaaaactgcgtctttgctgataatatcaaagt

SEQ ID NO: 33 - DNA, 67 bp, Artificial Sequence
  HBB pre-PC composition (i), pre-PC TO-1
ccttcagttagctttgatcttgtttatttcttgtcttctgctaggagggctttaagactttttgatg

SEQ ID NO: 34 - DNA, 64 bp, Artificial Sequence
  HBB pre-PC composition (i), pre-PC TO-2
cggaatctctgggacacagcttagacagtgttaagagagcccacatcaaaaagtcttaaagccc

SEQ ID NO: 35 - DNA, 57 bp, Artificial Sequence
  PSX pre-PC composition (i), pre-PC TO-1
agccagtcctgatagcatcagaaacccacatgtttcgggcaccaggttcacccactc

SEQ ID NO: 36 - DNA, 59 bp, Artificial Sequence
  PSX pre-PC composition (i), pre-PC TO-2
ttctgcacatcacattcatccacacccatccatcgcgccctgagagtgggtgaacctgg

SEQ ID NO: 37 - DNA, 60 bp, Artificial Sequence
  CT pre-PC composition (ii), pre-PC TO-1
tgataatatcaaagttggcaaccaatgcttctattacaattggtttggatgcggaaaaag

SEQ ID NO: 38 - DNA, 60 bp, Artificial Sequence
  CT pre-PC composition (ii), pre-PC TO-2
ttaaagctcaaaataaaaaagagtttatttttgggaaattcactgaaaactgcgtctttg

SEQ ID NO: 39 - DNA, 41 bp, Artificial Sequence
  CT pre-PC composition (ii), pre-PC LO-1
gttgccaactttgatattatcagcaaagacgcagttttcag

SEQ ID NO: 40 - DNA, 57 bp, Artificial Sequence
  HBB pre-PC composition (ii), pre-PC TO-1
tcttaaagccctcctagcagaagacaagaaataaacaagatcaaagctaactgaagg

SEQ ID NO: 41 - DNA, 52 bp, Artificial Sequence
  HBB pre-PC composition (ii), pre-PC TO-2
cggaatctctgggacacagcttagacagtgttaagagagcccacatcaaaaa

SEQ ID NO: 42 - DNA, 39 bp, Artificial Sequence
  HBB pre-PC composition (ii), pre-PC LO-1
tgctaggagggctttaagactttttgatgtgggctctct

SEQ ID NO: 43 - DNA, 48 bp, Artificial Sequence
  PSX pre-PC composition (ii), pre-PC TO-1
aacctggtgcccgaaacatgtgggtttctgatgctatcaggactggct

SEQ ID NO: 44 - DNA, 50 bp, Artificial Sequence
  PSX pre-PC composition (ii), pre-PC TO-2
ttctgcacatcacattcatccacacccatccatcgcgccctgagagtggg

SEQ ID NO: 45 - DNA, 31 bp, Artificial Sequence; identical to SEQ ID NO: 16
  PSX pre-PC composition (ii), pre-PC LO-1